SEQUENCE LISTING (35 sequences)

SEQ ID NO: 1 - protein, 4 aa, Artificial Sequence
  ELP component sequence
VPGG

SEQ ID NO: 2 - protein, 4 aa, Artificial Sequence
  ELP component sequence
IPGG

SEQ ID NO: 3 - protein, 5 aa, Artificial Sequence
  ELP component sequence
  misc_feature (4)..(4): Xaa can be any naturally occurring or non- natural amino acid
VPGXG

SEQ ID NO: 4 - protein, 5 aa, Artificial Sequence
  ELP component sequence
AVGVP

SEQ ID NO: 5 - protein, 5 aa, Artificial Sequence
  ELP component sequence
  misc_feature (4)..(4): Xaa can be any naturally occurring or non- natural amino acid
IPGXG

SEQ ID NO: 6 - protein, 5 aa, Artificial Sequence
  ELP component sequence
IPGVG

SEQ ID NO: 7 - protein, 5 aa, Artificial Sequence
  ELP component sequence
  misc_feature (4)..(4): Xaa can be any naturally occurring or non- natural amino acid
LPGXG

SEQ ID NO: 8 - protein, 5 aa, Artificial Sequence
  ELP component sequence
LPGVG

SEQ ID NO: 9 - protein, 6 aa, Artificial Sequence
  ELP component sequence
VAPGVG

SEQ ID NO: 10 - protein, 8 aa, Artificial Sequence
  ELP component sequence
GVGVPGVG

SEQ ID NO: 11 - protein, 9 aa, Artificial Sequence
  ELP component sequence
VPGFGVGAG

SEQ ID NO: 12 - protein, 9 aa, Artificial Sequence
  ELP component sequence
VPGVGVPGG

SEQ ID NO: 13 - protein, 5 aa, Artificial Sequence
  ELP component sequence
  misc_feature (1)..(1): Xaa can be any naturally occurring or non- natural amino acid
XPGVG

SEQ ID NO: 14 - protein, 84 aa, Artificial
  PTH
SVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 15 - DNA, 252 bp, Artificial
  PTH
agcgtgagcgaaattcagctgatgcataacctgggcaaacatctgaacagcatggaacgcgtggaatggctgcgcaaaaaactgcaggatgtgcataactttgtggcgctgggcgcgccgctggcgccgcgcgatgcgggcagccagcgcccgcgcaaaaaagaagataacgtgctggtggaaagccatgaaaaaagcctgggcgaagcggataaagcggatgtgaacgtgctgaccaaagcgaaaagccag

SEQ ID NO: 16 - protein, 34 aa, Artificial
  PTH 1-34
SVSEIQLMHNLGKHLNSMERVEWLRKKLQDVHNF

SEQ ID NO: 17 - DNA, 102 bp, Artificial
  PTH 1-34
agcgtgagcgaaattcagctgatgcataacctgggcaaacatctgaacagcatggaacgcgtggaatggctgcgcaaaaaactgcaggatgtgcataacttt

SEQ ID NO: 18 - protein, 32 aa, Artificial
  PTH 1-32
SVSEIQLMHNLGKHLNSMERVEWLRKKLQDVH

SEQ ID NO: 19 - DNA, 90 bp, Artificial
  PTH 1-32
agcgtgagcgaaattcagctgatgcataacctgggcaaacatctgaacagcatggaacgcgtggaatggctgcgcaaaaaactgcaggat

SEQ ID NO: 20 - protein, 690 aa, Artificial
  PTH ELP1-120 fusion
SVSEIQLMHNLGKHLNSMERVEWLRKKLQDVHNFVALGAPLAPRDAGSQRPRKKEDNVLVESHEKSLGEADKADVNVLTKAKSQGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 21 - DNA, 2076 bp, Artificial
  PTH ELP1-120 fusion
agcgtgagcgaaattcagctgatgcataacctgggcaaacatctgaacagcatggaacgcgtggaatggctgcgcaaaaaactgcaggatgtgcataactttgtggcgctgggcgcgccgctggcgccgcgcgatgcgggcagccagcgcccgcgcaaaaaagaagataacgtgctggtggaaagccatgaaaaaagcctgggcgaagcggataaagcggatgtgaacgtgctgaccaaagcgaaaagccagggtgtaccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggctggccgtgataa

SEQ ID NO: 22 - protein, 640 aa, Artificial
  PTH(1-34) ELP1-120 fusion
SVSEIQLMHNLGKHLNSMERVEWLRKKLQDVHNFGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 23 - DNA, 1923 bp, Artificial
  PTH(1-34) ELP1-120 fusion
agcgtgagcgaaattcagctgatgcataacctgggcaaacatctgaacagcatggaacgcgtggaatggctgcgcaaaaaactgcaggatgtgcataactttgtaccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggctggccgtgataa

SEQ ID NO: 24 - protein, 640 aa, Artificial
  PTHrp(1-34) ELP1-120 fusion
AVSEHQLLHDKGKSIQDLRRRFFLHHLIAEIHTAGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 25 - DNA, 1926 bp, Artificial
  PTHrp(1-34) ELP1-120 fusion
gcggtgagcgaacatcagctgctgcatgataaaggcaaaagcattcaggatctgcgccgccgcttttttctgcatcatctgattgcggaaattcataccgcgggtgtaccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggctggccgtgataa

SEQ ID NO: 26 - protein, 764 aa, Artificial
  PTH(1-34) ELPbetaV2-144 fusion
SVSEIQLMHNLGKHLNSMERVEWLRKKLQDVHNFVPGVGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGWP

SEQ ID NO: 27 - DNA, 2298 bp, Artificial
  PTH(1-34) ELPbetaV2-144 fusion
agcgtgagcgaaattcagctgatgcataacctgggcaaacatctgaacagcatggaacgcgtggaatggctgcgcaaaaaactgcaggatgtgcataactttgtaccgggcgtgggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggctggccgtgataa

SEQ ID NO: 28 - protein, 639 aa, Artificial
  PTH(1-34) ELP4-120 fusion
SVSEIQLMHNLGKHLNSMERVEWLRKKLQDVHNFVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGWP

SEQ ID NO: 29 - DNA, 1923 bp, Artificial
  PTH(1-34) ELP4-120 fusion
agcgtgagcgaaattcagctgatgcataacctgggcaaacatctgaacagcatggaacgcgtggaatggctgcgcaaaaaactgcaggatgtgcataactttgtaccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggcgtgggtgttccgggcgtaggtgtcccaggtgtgggcgtaccgggcgttggtgttcctggtgtcggcgtgccgggctggccgtgataa

SEQ ID NO: 30 - protein, 638 aa, Artificial
  PTH(1-32) ELP1-120 fusion
SVSEIQLMHNLGKHLNSMERVEWLRKKLQDVHGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 31 - DNA, 1917 bp, Artificial
  PTH(1-32) ELP1-120 fusion
agcgtgagcgaaattcagctgatgcataacctgggcaaacatctgaacagcatggaacgcgtggaatggctgcgcaaaaaactgcaggatgtgcatgtaccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggcgtgggtgttccgggcgtgggtgttccgggtggcggtgtgccgggcgcaggtgttcctggtgtaggtgtgccgggtgttggtgtgccgggtgttggtgtaccaggtggcggtgttccgggtgcaggcgttccgggtggcggtgtgccgggctggccgtgataa

SEQ ID NO: 32 - protein, 762 aa, Artificial
  PTH(1-32) ELPbetaV2-144 fusion
SVSEIQLMHNLGKHLNSMERVEWLRKKLQDVHVPGVGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGWP

SEQ ID NO: 33 - DNA, 2292 bp, Artificial
  PTH(1-32) ELPbetaV2-144 fusion
agcgtgagcgaaattcagctgatgcataacctgggcaaacatctgaacagcatggaacgcgtggaatggctgcgcaaaaaactgcaggatgtgcatgtaccgggcgtgggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggcgtgggtgtcccgggcgccggtgtgcctggagtgggtgtaccgggcgccggcgtgccaggcgtcggcgtgccgggtgtgggagtgcctggcgccggtgtccccggggtgggtgtcccaggtgccggtgtgccgggctggccgtgataa

SEQ ID NO: 34 - protein, 600 aa, Artificial Sequence
  ELP1-120
VPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGG

SEQ ID NO: 35 - protein, 730 aa, Artificial Sequence
  ELPbetaV2-144
VPGVGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVGVPGAGVPGVGVPGAGVPGVGVPGVGVPGAGVPGVGVPGAGVPGVG

(19) United States
(12) Patent Application Publication
(10) Pub. No.: US 2022/0289812 A1
(43) Pub. Date: Sep. 15, 2022

(54) ELP FUSION PROTEINS COMPRISING PARATHYROID HORMONE FOR CONTROLLED AND SUSTAINED RELEASE

(71) Applicant: PHASEBIO PHARMACEUTICALS, INC., MALVERN, PA (US)

(72) Inventors: David James BALLANCE, MALVERN, PA (US); James JOWETT, MALVERN, PA (US)

(21) Appl. No.: 17/633,369

(22) PCT Filed: Aug. 7, 2020
(86) PCT No.: PCT/US2020/045427

§ 371 Date: Feb. 7, 2022

Related U.S. Application Data
(60) Provisional application No. 62/884,792, filed on Aug. 9, 2019.

(51) Int. Cl. C07K 14/635; C07K 14/78

(52) U.S. Cl. CPC C07K 14/635; C07K 14/78; A61K 38/00

(57) ABSTRACT

The present disclosure provides novel pharmaceutical compositions comprising a parathyroid hormone or a parathyroid hormone related protein and an elastin-like peptide. These pharmaceutical compositions provide sustained, long-lasting control of serum calcium levels, and may be used in treating osteoporosis and hypoparathyroidism.

### CROSS-REFERENCE TO RELATED APPLICATIONS

This application claims the benefit of U.S. Provisional Application No. 62/884,792 filed Aug. 9, 2019, the contents of which are hereby incorporated by reference in their entirety for all purposes.

### SEQUENCE LISTING

The Sequence Listing associated with this application is provided in text format in lieu of a paper copy, and is hereby incorporated by reference into the specification. The name of the text file containing the Sequence Listing is PHAS-038_01WO_SeqList_ST25.txt. The text file is about 77 KB, was recorded on Aug. 7, 2020, and is being submitted electronically via EFS-Web.

### FIELD OF INVENTION

The present disclosure relates to pharmaceutical formulations for sustained release, and methods for delivering a treatment regimen with the sustained release formulations.

### BACKGROUND

Osteoporosis is a disease in which the density and quality of bone are reduced. As bones become more porous and fragile, the risk of fracture is greatly increased. The loss of bone occurs silently and progressively, and often there are no symptoms until the first fracture occurs. One in 3 women and 1 in 5 men aged fifty years and over are at risk of an osteoporotic fracture. The most common fractures associated with osteoporosis occur at the hip, spine and wrist. The likelihood of these fractures occurring, particularly at the hip and spine, increases with age in both women and men. Vertebral (spinal) and hip fractures are especially concerning, and vertebral fractures can result in serious consequences, including loss of height, intense back pain and deformity (sometimes called Dowager's Hump). A hip fracture often requires surgery and may result in loss of independence or death.

Hypoparathyroidism is caused by inadequate parathyroid hormone production. This leads to decreased blood levels of calcium (hypocalcemia) and increased levels of blood phosphorus (hyperphosphatemia). The condition can be inherited, but can also result from surgical removal of the thyroid or parathyroid glands.

Current treatments for osteoporosis and hypoparathyroidism include calcium supplementation and therapeutics to modify bone formation and/or bone resorption in an attempt to reach normal homeostasis. Many of these therapeutics are limited in their ability to precisely control serum calcium levels, and some have a toxic effect with long-term use.

### SUMMARY OF THE INVENTION

The present disclosure provides novel parathyroid hormone (PTH) and parathyroid hormone related protein pharmaceutical compositions comprising an elastin-like protein (ELP). These pharmaceutical compositions provide long-lasting, sustained release for the treatment of disorders such as osteoporosis and hypoparathyroidism.

In some aspects the present disclosure provides a pharmaceutical composition comprising a parathyroid hormone (PTH) or a parathyroid hormone related protein (PTHrP) and an elastin-like peptide, wherein the elastin-like peptide (ELP) comprises repeating units of any one of SEQ ID NOs: 1-13. In some embodiments, the PTH is a full-length PTH (SEQ ID NO: 14).

In some embodiments, the PTH is a truncated PTH. In some embodiments, the PTH is truncated at the C-terminus. In some embodiments, the PTH is PTH(1-34) (SEQ ID NO: 16) or PTH(1-32) (SEQ ID NO: 18). In some embodiments, the PTH is a PTH analog (e.g. Shimizu et al. 2016).

In some embodiments, the PTHrP is a full-length PTHrP. In some embodiments, the PTHrP is a truncated PTHrP. In some embodiments, the PTHrP is truncated at the C-terminus. In some embodiments, the PTHrP is PTHrP (1-34). In some embodiments, the PTHrP is PTHrP (1-32).

In some embodiments, the ELP comprises structural repeats of SEQ ID NO: 3. In some embodiments, X is independently selected from valine, glycine, and alanine. In some embodiments, X is independently selected from V, G and A at a ratio of 5:3:2. In some embodiments, X is independently selected from valine and alanine. In some embodiments, X is independently selected from valine and alanine at a ratio of 6:3.

In some embodiments, the ELP comprises at least 90 repeating structural units. In some embodiments, ELP comprises at least 120 repeating structural units. In some embodiments, the ELP comprises the amino acid sequence of SEQ ID NO: 34. In some embodiments, the ELP comprises at least 144 repeating structural units. In some embodiments, the ELP comprises the amino acid sequence of SEQ ID NO: 35.

In some embodiments the PTH or PTHrP and ELP are in a fusion protein. In some embodiments, the amino acid sequence of the fusion protein is selected from: a) SEQ ID NO: 20;

### b) SEQ ID NO: 22; c) SEQ ID NO: 24; d) SEQ ID NO: 26; e) SEQ ID NO: 28; and f) SEQ ID NO: 30.

In some aspects, the present disclosure provides methods of increasing serum calcium levels in a subject comprising administering to a subject in need thereof the pharmaceutical composition of the disclosure.

In some aspects, the present disclosure provides methods of increasing bone formation in a subject, comprising to a subject in need thereof administering the pharmaceutical composition of the present disclosure.

In some aspects, the present disclosure provides methods of reducing bone absorption in a subject, comprising administering to a subject in need thereof the pharmaceutical composition of the present disclosure.

In some aspects, the present disclosure provides methods of treating, ameliorating, or delaying osteoporosis, comprising administering to a subject in need thereof the pharmaceutical composition of the present disclosure.

In some aspects, the present disclosure provides methods of treating, ameliorating, or delaying hypoparathyroidism, comprising administering to a subject in need thereof the pharmaceutical composition of the present disclosure.

In some embodiments, the pharmaceutical composition is administered subcutaneously. In some embodiments, the pharmaceutical composition is administered daily or weekly.

### BRIEF DESCRIPTION OF THE FIGURES

FIGS. 1-4 show the construction of full length parathyroid hormone ELP1-120 fusions. DNA encoding full length PTH was synthesized, digested with restriction enzymes XbaI/BsrGI, and then sub-cloned into plasmid pPE0003 to provide plasmid pPE9356, placing the PTH sequence on the N-terminus of the ELP1-120 sequence. FIG. 1 is the plasmid map of pPE0003. FIG. 2 is the plasmid map of pPE9356. FIG. 3 is the amino acid sequence of the fusion protein. FIG. 4 is the DNA sequence encoding the fusion protein.

FIGS. 5-7 show the construction of PTH (1-34) ELP1-120 fusions. DNA encoding the first 34 amino acids of the N-terminal sequence of PTH was synthesized, digested with restriction enzymes XbaI/BsrGI, and then sub-cloned into plasmid pPE0003 to provide plasmid pPE9366, placing the PTH(1-34) sequence on the N-terminus of the ELP1-120 sequence. FIG. 5 is the plasmid map of pPE9366. FIG. 6 is the amino acid sequence of the fusion protein. FIG. 7 is the DNA sequence encoding the fusion protein.

FIGS. 8-10 show the construction of PTHrp (1-34) ELP1-120 fusions. DNA encoding the first 34 amino acids of the N-terminal sequence of PTH related protein (PTHrp) was synthesized, digested with restriction enzymes XbaI/BsrGI, and then sub-cloned into plasmid pPE0003 to provide plasmid pPE9616, placing the PTHrp(1-34) sequence on the N-terminus of the ELP1-120 sequence. FIG. 8 is the plasmid map of pPE9616. FIG. 9 is the amino acid sequence of the fusion protein. FIG. 10 is the DNA sequence encoding the fusion protein.

FIGS. 11-14 show the construction of PTH (1-34) ELPbetaV2-144 fusions. DNA encoding the first 34 amino acids of the N-terminal sequence of PTH was synthesized, digested with restriction enzymes XbaI/BsrGI, and then sub-cloned into plasmid pPE0584 to provide plasmid pPE9636, placing the PTH(1-34) sequence on the N-terminus of the ELPbetaV2-144 sequence. FIG. 11 is the plasmid map of pPE0584. FIG. 12 is the plasmid map of pPE9636. FIG. 13 is the amino acid sequence of the fusion protein. FIG. 14 is the DNA sequence encoding the fusion protein.

FIGS. 15-18 show the construction of PTH(1-34) ELP4-120 fusions. DNA encoding the first 34 amino acids of the N-terminal sequence of PTH was synthesized, digested with restriction enzymes XbaI/BsrGI, and then sub-cloned into plasmid pPE0678 to provide plasmid pPE9267, placing the PTH(1-34) sequence on the N-terminus of the ELP4-120 sequence. FIG. 15 is the plasmid map of pPE0678. FIG. 16 is the plasmid map of pPE9267. FIG. 17 is the amino acid sequence of the fusion protein. FIG. 18 is the DNA sequence encoding the fusion protein.

FIGS. 19-21 show the construction of PTH(1-32) ELP1-120 fusions. DNA encoding the first 32 amino acids of the N-terminal sequence of PTH was synthesized, digested with restriction enzymes XbaI/BsrGI, and then sub-cloned into plasmid pPE0003 to provide plasmid pPE9277, placing the PTH(1-32) sequence on the N-terminus of the ELP1-120 sequence. FIG. 19 is the plasmid map of pPE9277. FIG. 20 is the amino acid sequence of the fusion protein. FIG. 21 is the DNA sequence encoding the fusion protein.

FIGS. 22-24 show the construction of PTH(1-32) ELPbetaV2-144 fusions. DNA encoding the first 32 amino acids of the N-terminal sequence of PTH was synthesized, digested with restriction enzymes XbaI/BsrGI, and then sub-cloned into plasmid pPE0584 to provide plasmid pPE9287, placing the PTH(1-32) sequence on the N-terminus of the ELPbetaV2-144 sequence. FIG. 22 is the plasmid map of pPE9287. FIG. 23 is the amino acid sequence of the fusion protein. FIG. 24 is the DNA sequence encoding the fusion protein.

FIG. 25-26 show results of potency determinations of PE9366 and PE9636. FIG. 25 shows potency based on cAMP generated upon activation of the PTHR1 receptor using a Eurofins cAMP Hunter™ eXpress PTHR1 CHO-K1 GPCR assay kit. FIG. 26 shows potency based on the recruitment of β-arrestin 2 following binding to the PTHR1 receptor using a Eurofins (DiscoverX) PathHunter eXpress PTHR1 CHO-K1 β-Arrestin GPCR Assay kit.

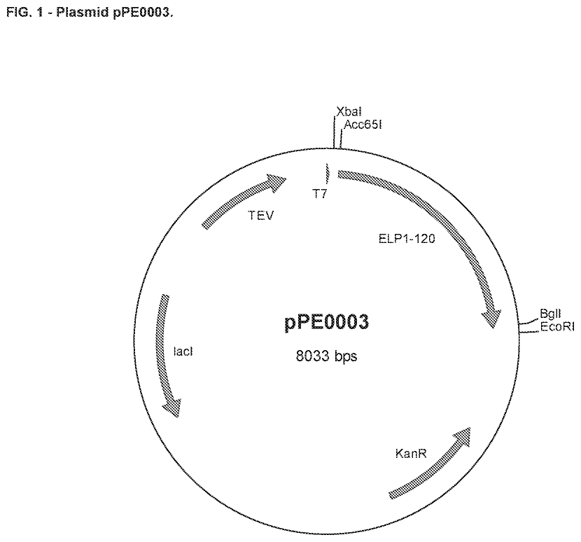

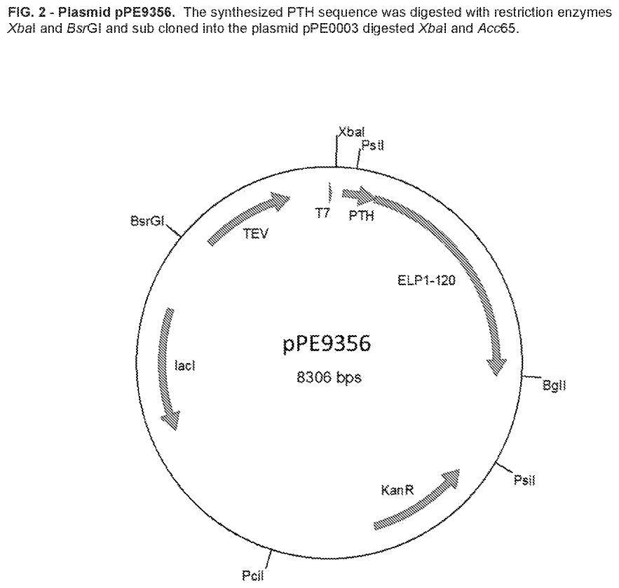

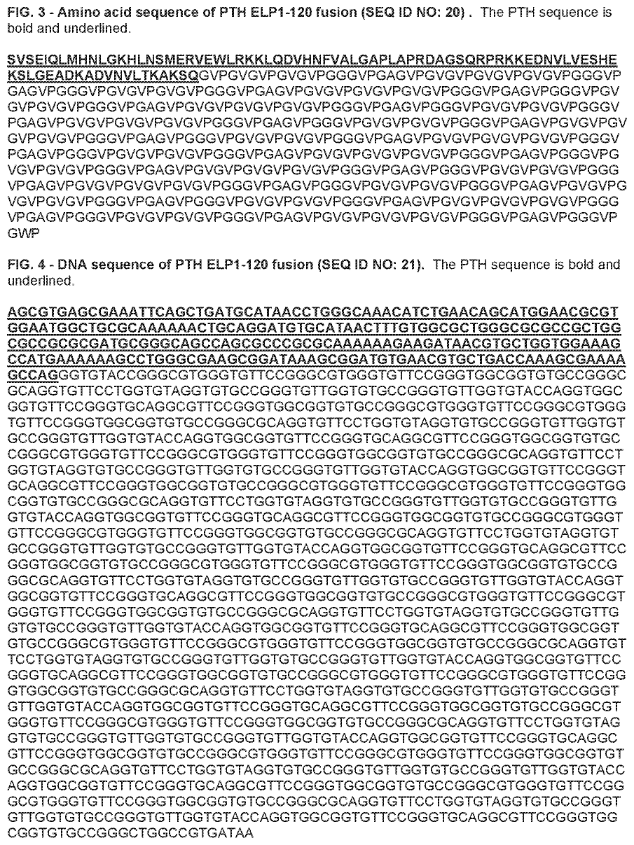

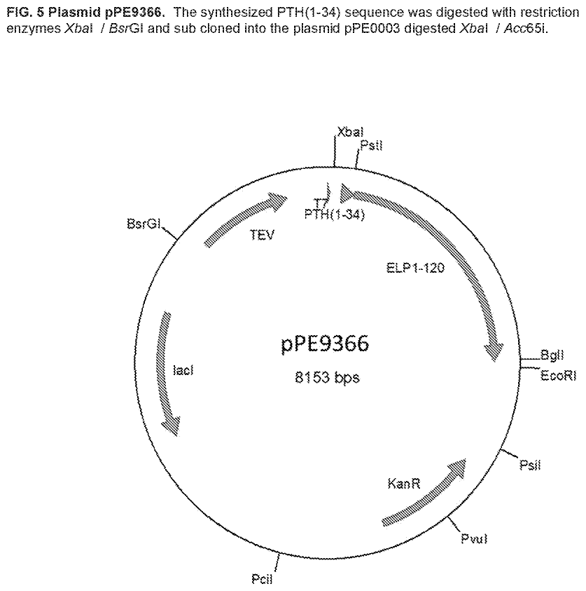

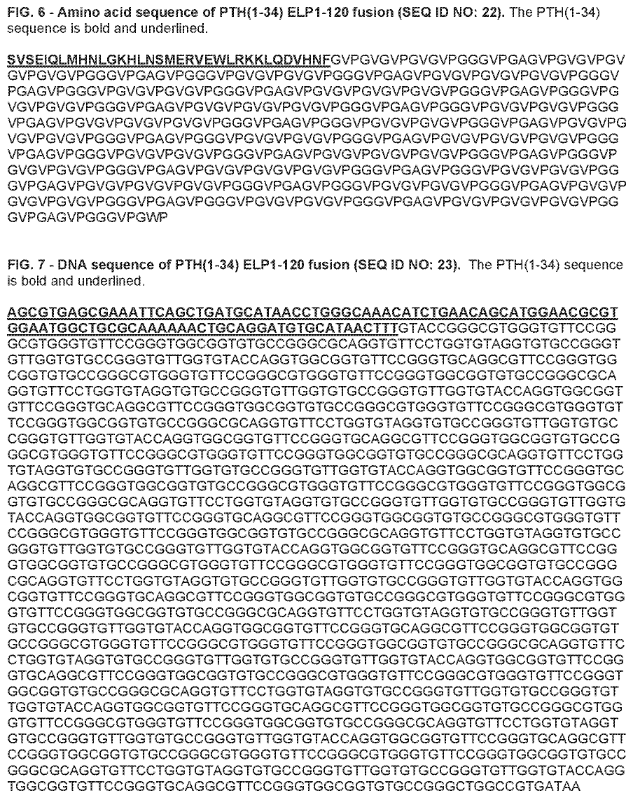

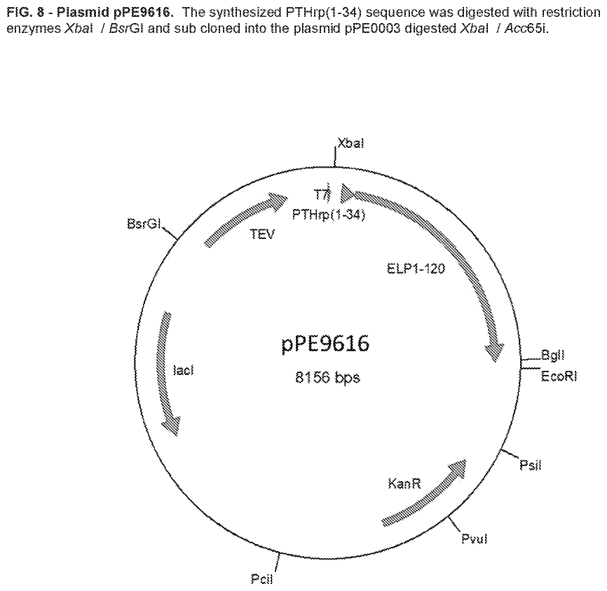

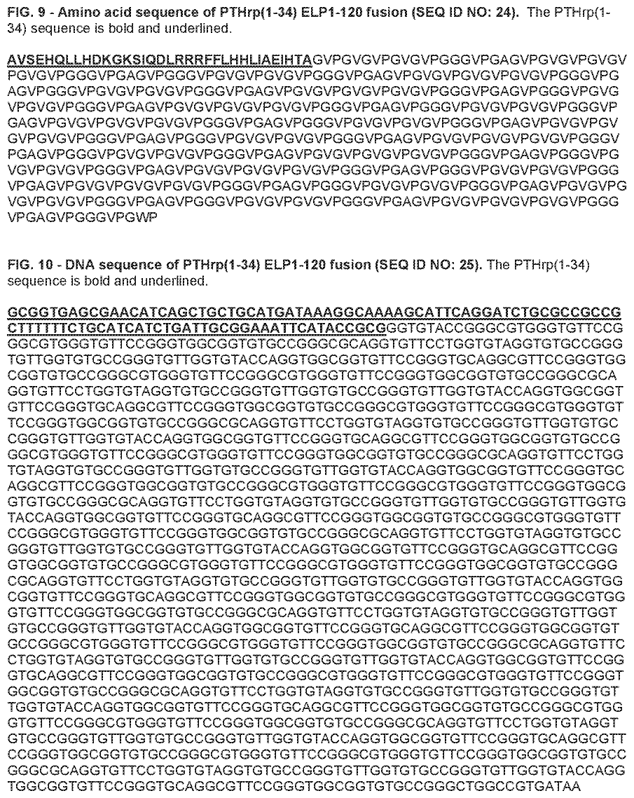

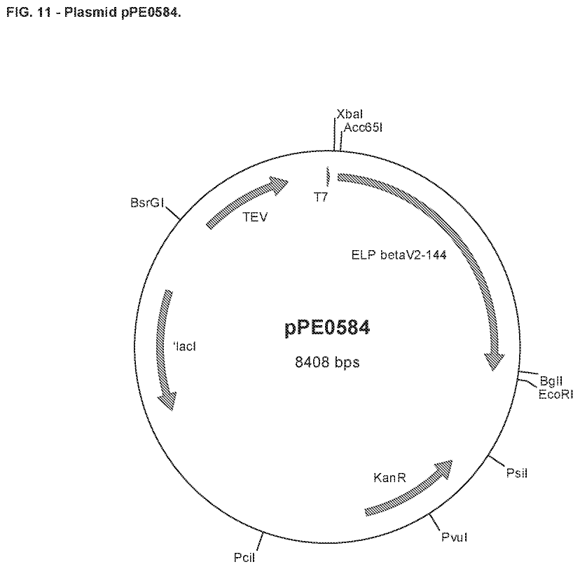

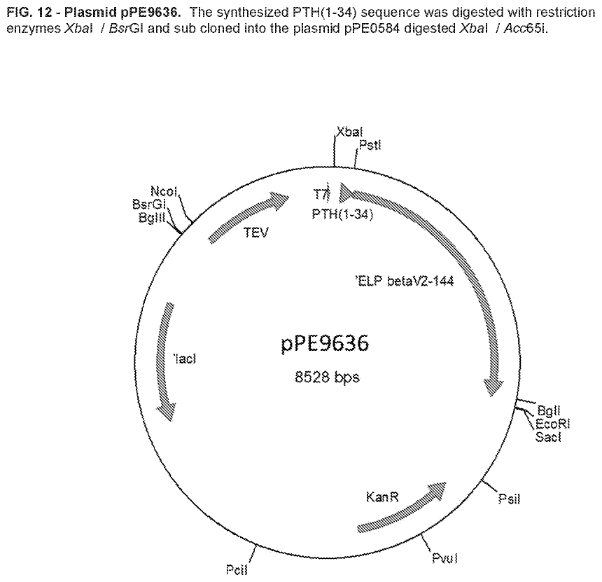

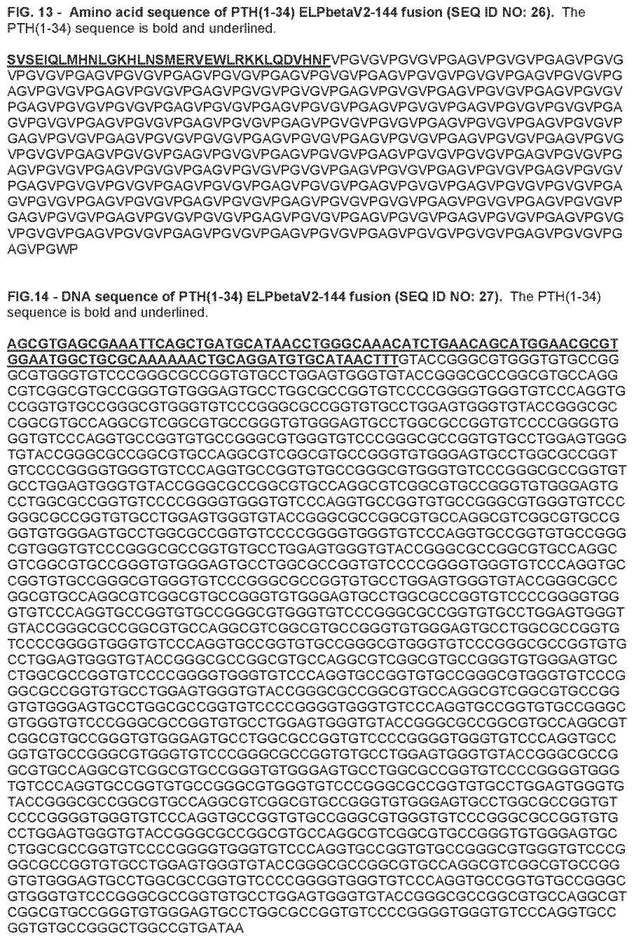

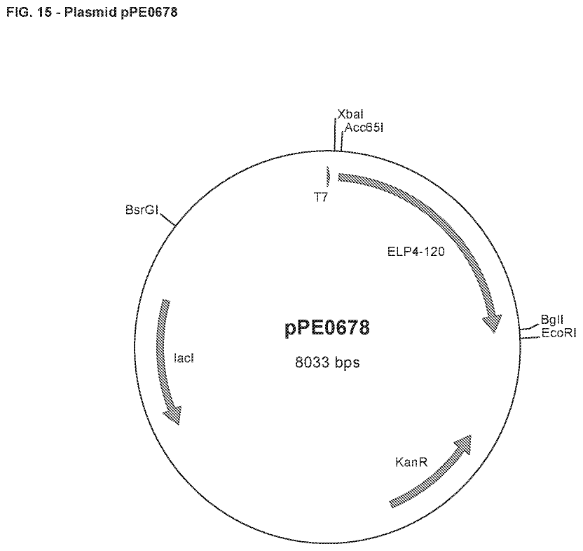

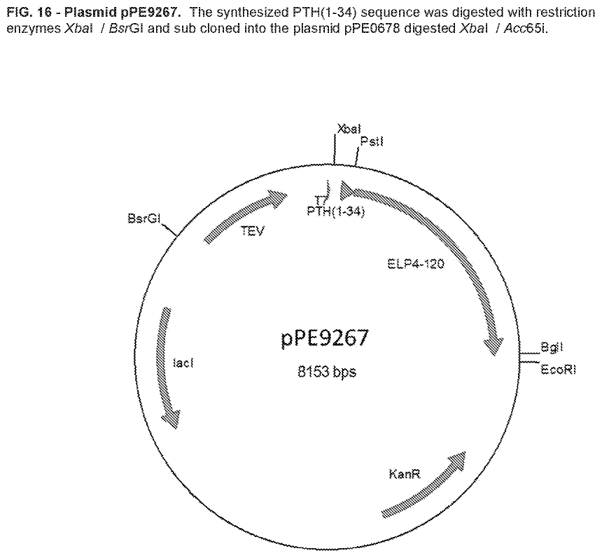

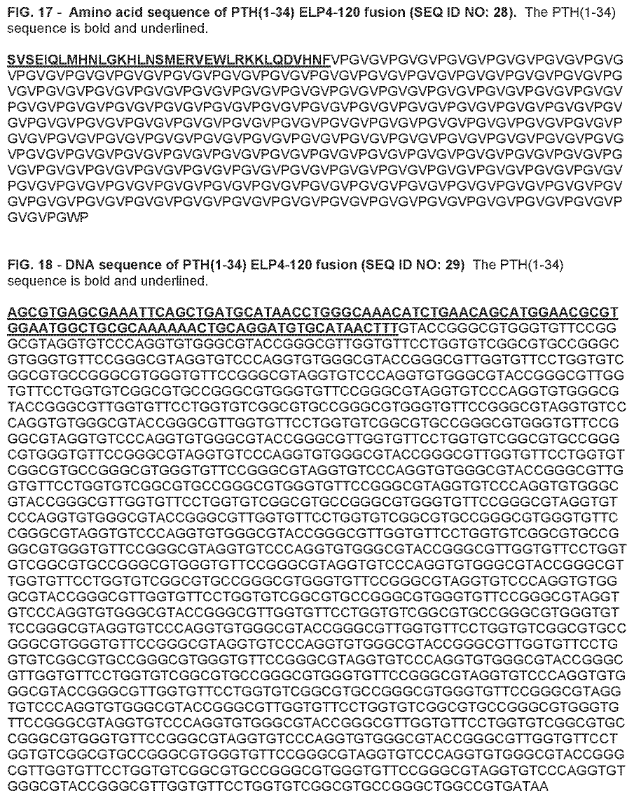

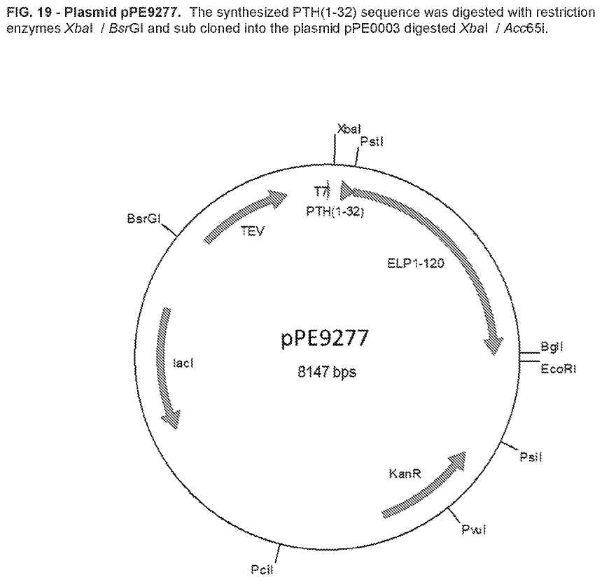

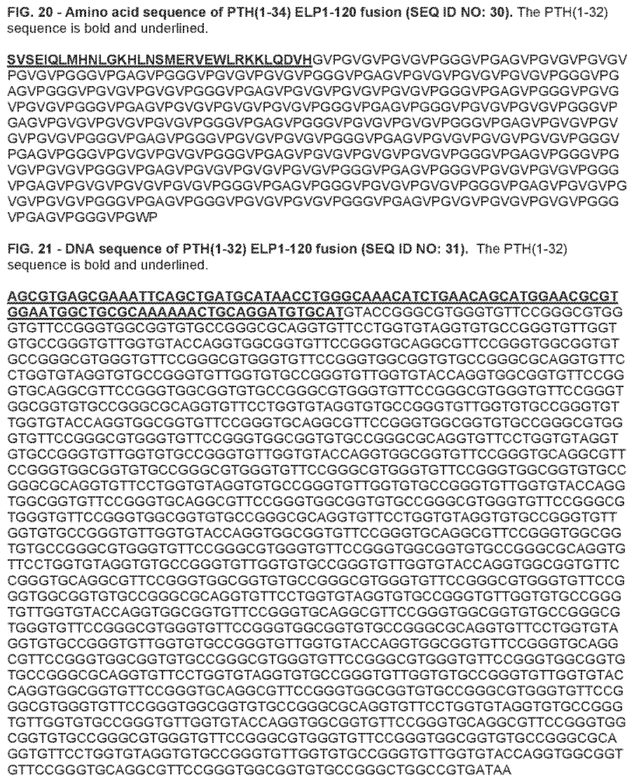

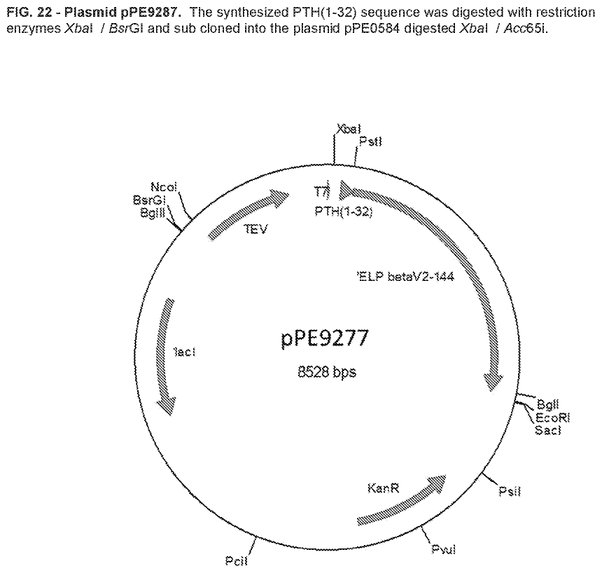

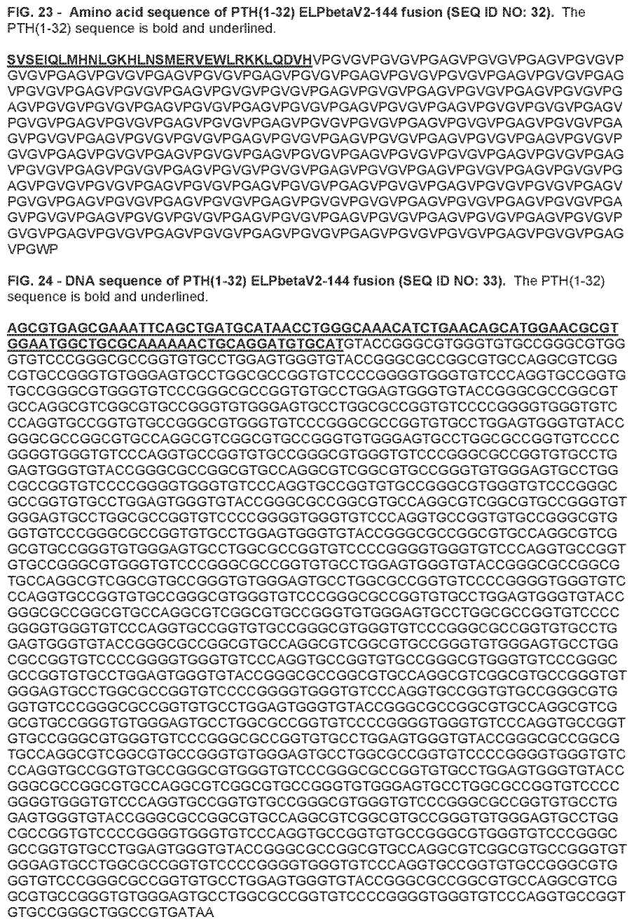

### DETAILED DESCRIPTION

The present disclosure provides novel pharmaceutical compositions comprising a parathyroid hormone (PTH) or a parathyroid hormone related protein (PTHrP) and an elastin-like protein (ELP). These compositions provide sustained, long-lasting control of serum calcium levels and bone formation in patients.

### Parathyroid Hormone and Parathyroid Hormone Related Protein

Parathyroid hormone (PTH) is a peptide hormone secreted from the parathyroid glands and is involved in the regulation of serum calcium levels, through effects on bone resorption, renal tubular calcium reabsorption and increasing intestinal calcium absorption. PTH is secreted as an 84-amino acid peptide in response to decreases in calcium concentration. Teriparatide (PTH 1-34) is equipotent to the full length peptide and is approved in the United States for treatment of osteoporosis in those at high risk of fracture including postmenopausal women, men with primary or hypogonadal osteoporosis and men and women with glucocorticoid-associated osteoporosis. Full length PTH (1-84) is approved in Europe for use in postmenopausal women with osteoporosis, but has been reported to lead to a higher frequency of hypercalcemia and hypercalciuria than teriparatide. Full length PTH (1-84) (NATPARA) is approved in US as an adjunct to calcium and vitamin D to control hypocalcemia in patients with hypoparathyroidism.

However, the use of these therapeutics is limited by the deleterious effects of cumulative administration, such as osteosarcoma. Further, these therapeutics have a short half-life (e.g. 75 minutes for PTH (1-34)), thus requiring frequent administration in an attempt to maintain proper serum calcium levels. Novel, long-lasting forms of PTH and PTHrP polypeptides are required.

The present application discloses these peptides, and a novel PTH 1-32 in fusion with an elastin-like peptide. The present inventors have found that, contrary to what is known in the art, the PTH 1-32 polypeptide is active.

PTH/PTHrP receptor 1 (PTHR1)

PTH and PTHrP bind to and activate a family B G-protein-coupled receptor known as PTH/PTHrP type 1 receptor (PTHR1), though the biological functions of the two ligands are distinct since PTH acts in an endocrine manner on bone and kidney cells to regulate blood calcium and phosphate, whereas PTHrP acts in a paracrine manner within developing tissues, such as the skeletal growth plate. Activation of the receptor leads to signaling via Gas-cAMP, via Gα_{q}-PLC-β signaling or via the β-arrestin-ERK1/2 pathway. The receptor also exists in discrete conformations, either coupled to heterotrimeric G protein (R^{G} conformation) or not coupled to a G protein (R⁰ conformation). Analogs of PTH or PTHrP may bind to or activate different forms of the receptor, so-called biased agonists. For instance, replacing isoleucine at position 5 in PTH with histidine leads to significant reduction in binding to the R⁰ conformation of the receptor, whereas replacing histidine at position 5 of PTHrP with isoleucine significantly enhances binding of PTHrP to this conformation of the receptor (Dean et al., 2008).

### PTH Receptor 2 (PTHR1)

Human PTH2R shares only 51% amino acid sequence identity with human PTH1R. PTH binds and stimulates both receptors, whereas PTHrP effects on only PTH1R. The endogenous ligand tuberoinfundibular peptide of 39 residues (TIP39) also binds to both receptors, but its affinity to the PTH2R a hundredfold stronger than to PTH1R. TIP39 has an antagonistic activity on PTH1R.

### Parathyroid Hormone

Parathyroid hormone and related peptides have both anabolic and catabolic functions. Administration of PTH increases markers for both bone formation and bone resorption. PTH mobilizes calcium and phosphorus into circulation by stimulating bone resorption, and maintains serum calcium levels.

In certain embodiments of the disclosure, the pharmaceutical composition includes an amino acid sequence providing sustained release fused or conjugated to parathyroid hormone (PTH), mimetics, analogs, derivatives, fragments, or functional variants thereof. In some embodiments, the amino acid sequence providing sustained release is an ELP. In certain embodiments, the PTH is a mammalian PTH. In some embodiments, the PTH is human PTH (e.g., SEQ ID NO: 14), which is the full length mature human PTH peptide, PTH 1-84 (SEQ ID NO: 14). In other embodiments, the PTH is a truncation of PTH including, but not limited to, PTH(1-34) (SEQ ID NO: 16) and PTH(1-32) (SEQ ID NO: 18). In some embodiments, the PTH includes one or more modified amino acids. In some embodiments, the PTH includes one or more amino acid derivatives.

In some embodiments, the PTH is a functional analog of mammalian PTH, including functional fragments truncated at the N-terminus and/or the C-terminus of PTH by from about 1 to about 30 amino acids, including, for example, by up to about 3 amino acids, up to about 5 amino acids, up to about 10 amino acids, up to about 15 amino acids, up to about 20 amino acids, up to about 25 amino acids, or up to about 30 amino acids. In other embodiments, functional variants contain from about 1 to about 30 amino acid insertions, deletions, and/or substitutions with respect to a native or truncated sequence (e.g., SEQ ID NOs: 14, 16, or 18). For example, functional variants may have up to about 3 amino acid, up to about 5 amino acid, up to about 10 amino acid, up to about 15 amino acid, up to about 20 amino acid, up to about 25 amino acid, or up to about 30 amino acid insertions, deletions, and/or substitutions with respect to a native or truncated sequence (e.g., SEQ ID NOs: 14, 16, or 18). Protein activity may be confirmed or assayed using any available assay. In other embodiments, the PTH has at least about 75% identity, about 80% identity, about 90% identity, about 95% identity, about 96% identity, about 97% identity, about 98% identity, or about 99% identity with a native or truncated sequence (e.g., SEQ ID NOs: 14, 16, or 18). Percentage identity can be calculated using the alignment program EMBOSS needle, available at http://www.ebi.ac.uk/Tools/psa/emboss_needle/. The following default parameters may be used for Pairwise alignment: Protein Weight Matrix=BLOSUM62; Gap Open=10; Gap Extension=0.1. In some embodiments, the PTH may contain additional chemical modifications known in the art. In some embodiments, the PTH functional analog preferentially binds to the R⁰ conformation of the PTHR1. In some embodiments, the PTH functional analog preferentially binds to the R^{G} conformation of the PTHR1. In some embodiments, the PTH functional analog activates the receptor in such a way as to preferentially signal via Gas-cAMP. In some embodiments, the PTH functional analog activates the receptor in such a way as to preferentially signal via Gα_{q}-PLC-β. In some embodiments, the PTH functional analog activates the receptor in such a way as to preferentially signal via the β-arrestin-ERK1/2 pathway.

In some embodiments, the present disclosure provides nucleic acid sequences encoding an amino acid sequence providing sustained release fused or conjugated to parathyroid hormone (PTH), mimetics, analogs, derivatives, fragments, or functional variants thereof. In certain embodiments, the nucleic acid sequence encodes a mammalian PTH. In some embodiments, the nucleic acid sequence encodes a full length mature human PTH (e.g., SEQ ID NO: 15). In other embodiments, the nucleic acid sequence encodes a truncated PTH including, but not limited to, PTH(1-34) (SEQ ID NO: 17) and PTH(1-32) (SEQ ID NO: 19). In some embodiments, the nucleic acid encodes a PTH including one or more modified amino acids. In some embodiments, the nucleic acid encodes a PTH including one or more amino acid derivatives.

In some embodiments, the nucleic acid encodes a PTH that is a functional analog of mammalian PTH, including functional fragments truncated at the N-terminus and/or the C-terminus of PTH by from about 1 to about 30 amino acids, including, for example, by up to about 3 amino acids, up to about 5 amino acids, up to about 10 amino acids, up to about 15 amino acids, up to about 20 amino acids, up to about 25 amino acids, or up to about 30 amino acids. In other embodiments, functional variants contain from about 1 to about 90 nucleic acid insertions, deletions, and/or substitutions with respect to a native or truncated sequence (e.g., SEQ ID NOs: 15, 17, or 19). For example, functional variants may have up to about 9 nucleic acids, up to about 15 nucleic acids, up to about 30 nucleic acids, up to about 45 nucleic acids, up to about 60 nucleic acid, up to about 75 nucleic acid, or up to about 90 nucleic acid insertions, deletions, and/or substitutions with respect to a native or truncated sequence (e.g., SEQ ID NOs: 15, 17, or 19). Protein activity may be confirmed or assayed using any available assay. In other embodiments, the PTH nucleic acid has at least about 75% identity, about 80% identity, about 90% identity, about 95% identity, about 96% identity, about 97% identity, about 98% identity, or about 99% identity with a nucleic acid encoding a native or truncated sequence (e.g., SEQ ID NOs: 15, 17, or 19). Percentage identity can be calculated using the alignment program EMBOSS needle, available at http://www.ebi.ac.uk/Tools/psa/emboss_needle/. In some embodiments, the nucleic acid is DNA, RNA, mRNA, or a combination thereof.

In certain embodiments, the therapeutic agent includes an ELP fused to the N-terminus or the C-terminus of PTH, mimetics, analogs, derivatives, fragments, or functional variants thereof. In some aspects, the therapeutic agent includes an ELP fused to the C-terminus of human full length PTH (SEQ ID NO: 14). In some embodiments, the PTH is in a fusion protein with more than one ELP sequence. In some embodiments, the PTH has one or more ELPs at both the N- and C-termini. In some embodiments, the ELP at the C-terminus and/or the N-terminus of the PTH includes about 90 to about 180 repeating structural units. In some embodiments, the ELP at the C-terminus and/or the N-terminus of the PTH includes about 90, about 120, or about 144 repeating structural units. In other embodiments, the ELP at the C-terminus and/or the N-terminus of the PTH includes fewer than about 90 repeating structural units. In other aspects, the ELP at the C-terminus and/or the N-terminus of the PTH includes greater than about 180 repeating structural units. In some embodiments, the two or more ELPs at the N- and C-termini are approximately the same size. In other embodiments, the two or more ELPs at the N- and C-termini differ in size. In some embodiments, the ELP at the N-terminus of the PTH is larger than the ELP at the C-terminus of the PTH. In other embodiments, the ELP at the C-terminus of the PTH is larger than the ELP at the N-terminus of the PTH.

### Parathyroid Hormone Related Protein

Parathyroid Hormone Related Protein (PTHrP) binds to the PTH-1 receptor, and activates signal transduction with an equal potency as PTH. Administration of PTHrP results in an equivalent increase in lumbar spine bone mineral density (BMD) as PTH. Unlike PTH however, PTHrP has a purely anabolic effect, and lacks the concomitant increase in bone resorption observed with PTH.

In certain embodiments of the disclosure, the pharmaceutical composition includes an amino acid sequence providing sustained release fused or conjugated to parathyroid hormone related protein (PTHrP), mimetics, analogs, derivatives, fragments, or functional variants thereof. In some embodiments, the amino acid sequence providing sustained release is an ELP. In certain embodiments, the PTHrP is a mammalian PTH. In some embodiments, the PTHrP is human full length PTHrP. In other embodiments, the PTHrP is a truncation of PTHrP including, but not limited to, PTHrP(1-34). In some embodiments, the PTHrP includes one or more modified amino acids. In some embodiments, the PTHrP includes one or more amino acid derivatives.

In some embodiments, the PTHrP is a functional analog of mammalian PTHrP, including functional fragments truncated at the N-terminus and/or the C-terminus of PTHrP by from about 1 to about 30 amino acids, including, for example, by up to about 3 amino acids, up to about 5 amino acids, up to about 10 amino acids, up to about 15 amino acids, up to about 20 amino acids, up to about 25 amino acids, or up to about 30 amino acids. In other embodiments, functional variants contain from about 1 to about 30 amino acid insertions, deletions, and/or substitutions with respect to a native or truncated sequence. For example, functional variants may have up to about 3 amino acid, up to about 5 amino acid, up to about 10 amino acid, up to about 15 amino acid, up to about 20 amino acid, up to about 25 amino acid, or up to about 30 amino acid insertions, deletions, and/or substitutions with respect to a native or truncated sequence. Protein activity may be confirmed or assayed using any available assay. In other embodiments, the PTHrP has at least about 75% identity, about 80% identity, about 90% identity, about 95% identity, about 96% identity, about 97% identity, about 98% identity, or about 99% identity with a native or truncated sequence. Percentage identity can be calculated using the alignment program EMBOSS needle, available at http://www.ebi.ac.uk/Tools/psa/emboss_needle/. The following default parameters may be used for Pairwise alignment: Protein Weight Matrix=BLOSUM62; Gap Open=10; Gap Extension=0.1. In some embodiments, the PTHrP may contain additional chemical modifications known in the art. In some embodiments, the PTHrP functional analog preferentially binds to the R⁰ conformation of the PTHR1. In some embodiments, the PTHrP functional analog preferentially binds to the R^{G} conformation of the PTHR1. In some embodiments, the PTHrP functional analog activates the receptor in such a way as to preferentially signal via Gas-cAMP. In some embodiments, the PTHrP functional analog activates the receptor in such a way as to preferentially signal via Gα_{q}-PLC-β. In some embodiments, the PTHrP functional analog activates the receptor in such a way as to preferentially signal via the β-arrestin-ERK1/2 pathway.

In some embodiments, the analog of PTHrP comprises substitutions that confer stability to the polypeptide. In some embodiments, the PTHrP analog is identical to PTHrP for a portion of the sequence, and contains another portion including substantial substitutions. In some embodiments, the analog of PTHrP is a polypeptide that is identical to the amino acid sequence of PTHrP at amino acids 1 to 22, but then contains substitutions between amino acids 23 to 34. In some embodiments, the PTHrP analog is Abalopartide.

In some embodiments, the present disclosure provides nucleic acid sequences encoding an amino acid sequence providing sustained release fused or conjugated to parathyroid hormone related protein related protein (PTHrP), mimetics, analogs, derivatives, fragments, or functional variants thereof. In certain embodiments, the nucleic acid encodes a mammalian PTHrP. In some embodiments, the nucleic acid encodes a full length mature human PTHrP peptide. In other embodiments, the nucleic acid encodes a truncated PTHrP including, but not limited to, PTHrP(1-34). In some embodiments, the nucleic acid encodes PTHrP including one or more modified amino acids. In some embodiments, the nucleic acid encodes a PTHrP including one or more amino acid derivatives.

In some embodiments, the nucleic acid encodes a PTHP that is a functional analog of mammalian PTHrP, including functional fragments truncated at the N-terminus and/or the C-terminus of PTHrP by from about 1 to about 30 amino acids, including, for example, by up to about 3 amino acids, up to about 5 amino acids, up to about 10 amino acids, up to about 15 amino acids, up to about 20 amino acids, up to about 25 amino acids, or up to about 30 amino acids. In other embodiments, the nucleic acid sequence encoding functional variants contains from about 1 to about 90 nucleic acid insertions, deletions, and/or substitutions with respect to a native or truncated sequence. For example, nucleic acid sequences encoding functional variants may have up to about 9 nucleic acids, up to about 15 nucleic acids, up to about 30 nucleic acids, up to about 45 nucleic acids, up to about 60 nucleic acid, up to about 75 nucleic acid, or up to about 90 nucleic acid insertions, deletions, and/or substitutions with respect to a native or truncated sequence. Protein activity may be confirmed or assayed using any available assay. In other embodiments, the nucleic acid sequence encoding PTHrP has at least about 75% identity, about 80% identity, about 90% identity, about 95% identity, about 96% identity, about 97% identity, about 98% identity, or about 99% identity with a nucleic acid sequence encoding a native or truncated sequence. Percentage identity can be calculated using the alignment program EMBOSS needle, available at http://www.ebi.ac.uk/Tools/psa/emboss_needle/. In some embodiments, the nucleic acid is DNA, RNA, mRNA, or a combination thereof.

In certain embodiments, the therapeutic agent includes an ELP fused to the N-terminus or the C-terminus of PTHrP, mimetics, analogs, derivatives, fragments, or functional variants thereof. In some aspects, the therapeutic agent includes an ELP fused to the C-terminus of human full length PTHrP. In some embodiments, the PTHrP is in a fusion protein with more than one ELP sequence. In some embodiments, the PTHP has one or more ELPs at both the N- and C-termini. In some embodiments, the ELP at the C-terminus and/or the N-terminus of the PTHrP includes about 90 to about 180 repeating structural units. In some embodiments, the ELP at the C-terminus and/or the N-terminus of the PTHrP includes about 90, about 120, or about 144 repeating structural units. In other embodiments, the ELP at the C-terminus and/or the N-terminus of the PTHrP includes fewer than about 90 repeating structural units. In other aspects, the ELP at the C-terminus and/or the N-terminus of the PTHrP includes greater than about 180 repeating structural units. In some embodiments, the two or more ELPs at the N- and C-termini are approximately the same size. In other embodiments, the two or more ELPs at the N- and C-termini differ in size. In some embodiments, the ELP at the N-terminus of the PTHP is larger than the ELP at the C-terminus of the PTHrP. In other embodiments, the ELP at the C-terminus of the PTHrP is larger than the ELP at the N-terminus of the PTHrP.

### Elastin-Like Peptides

The present disclosure provides pharmaceutical formulations for sustained release, and methods for delivering a treatment regimen with the sustained release formulations. In certain embodiments, the pharmaceutical compositions disclosed herein have enhanced efficacy, bioavailability, circulatory half-life, persistence, degradation resistance, etc. The disclosure thereby provides improved pharmacokinetics for active agents, such as peptides and small molecule drugs, including a relatively flat PK profile with a low ratio of peak to trough, and/or a long Tmax. The PK profile can be maintained with a relatively infrequent administration schedule, such as from one to eight injections per month in some embodiments.

In some aspects, the disclosure provides sustained release pharmaceutical formulations. The formulation includes therapeutic agents for systemic administration, where the therapeutic agent includes an active agent and an amino acid sequence capable of forming a matrix or coacervate at the body temperature of a subject. The reversible matrix is formed from hydrogen bonds (e.g., intra- and/or intermolecular hydrogen bonds) as well as from hydrophobic contributions. The formulation further includes one or more pharmaceutically acceptable excipients and/or diluents. The matrix provides for a slow absorption to the circulation from an injection site. Without being bound by theory, this slow absorption is due to the slow reversal of the matrix or coacervate at the periphery of the injection site depot. The slow absorption profile provides for a flat PK profile, as well as convenient and comfortable administration regimen. For example, in various embodiments, the plasma concentration of the active agent over the course of days (e.g., from 2 to about 60 days, or from about 4 to about 30 days) does not change by more than a factor of 20, or by more than a factor of about 10, or by more than a factor of about 5, or by more than a factor of about 3. Generally, this flat PK profile is seen over a plurality of (substantially evenly spaced) administrations, such as at least about 2, at least about 5, or at least about 10 administrations of the formulation. In some embodiments, the slow absorption is manifest by a Tmax (time to maximum plasma concentration) of greater than about 5 hours, greater than about 10 hours, greater than about 20 hours, greater than about 30 hours, or greater than about 50 hours.

### Amino Acid Sequences Forming a Reversible Matrix

The sustained release, or slow absorption from the injection site, is controlled by the amino acid sequence capable of forming a hydrogen-bonded matrix or coacervate at the body temperature of the subject.

In some embodiments, the amino acid sequence contains structural units that form hydrogen-bonds through protein backbone groups and/or side chain groups, and which may contribute hydrophobic interactions to matrix formation. In some embodiments, the amino acid side chains do not contain hydrogen bond donor groups, with hydrogen bonds being formed substantially through the protein backbone. Exemplary amino acids include proline, alanine, valine, glycine, and isoleucine, and similar amino acids. In some embodiments, the structural units are substantially repeating structural units, so as to create a substantially repeating structural motif, and substantially repeating hydrogen-bonding capability. In these and other embodiments, the amino acid sequence contains at least about 10%, at least about 20%, at least about 40%, or at least about 50% proline, which may be positioned in a substantially repeating pattern. In this context, a substantially repeating pattern means that at least about 50% or at least about 75% of the proline residues of the amino acid sequence are part of a definable structural unit. In still other embodiments, the amino acid sequence contains amino acids with hydrogen-bond donor side chains, such as serine, threonine, and/or tyrosine. In some embodiments, the repeating sequence may contain from one to about four proline residues, with remaining residues independently selected from non-polar residues, such as glycine, alanine, leucine, isoleucine, and valine. Non-polar or hydrophobic residues may contribute hydrophobic interactions to the formation of the matrix.

In other embodiments, the amino acid sequence capable of forming the matrix at body temperature may include a random coil or non-globular extended structure. For example, the amino acid sequence capable of forming the matrix at body temperature may comprise an amino acid sequence disclosed in U.S. Patent Publication No. 2008/0286808, WIPO Patent Publication No. 2008/155134, and U.S. Patent Publication No. 2011/0123487, each of which is hereby incorporated by reference.

In some embodiments the amino acid sequence includes an unstructured recombinant polymer of at least 40 amino acids. The unstructured polymer may include more than about 100, about 150, about 200 or more contiguous amino acids. In some embodiments, the amino acid sequence forms a random coil domain. In particular, a polypeptide or amino acid polymer having or forming “random coil conformation” substantially lacks a defined secondary and tertiary structure. In some embodiments, the unstructured polymer is defined as a polymer having at least 40 amino acids where the total number of glycine (G), aspartate (D), alanine (A), serine (S), threonine (T), glutamate (E) and proline (P) residues constitutes more than about 80% of the total amino acids in the polymer. In some embodiments, at least 50% of the amino acids are devoid of secondary structure as determined by the Chou-Fasman algorithm.

The amino acid sequences may form a “gel-like” state upon injection at a temperature higher than the storage temperature. Exemplary sequences have repeating peptide units, and/or may be relatively unstructured at the lower temperature, and achieve a hydrogen-bonded, structured, state at the higher temperature.

### Elastin-Like Peptides (ELPs)

In some embodiments, the amino acid sequence capable of forming a matrix at body temperature is a peptide having repeating units of from four to ten amino acids. The repeating unit may form one, two, or three hydrogen bonds in the formation of the matrix. In certain embodiments, the amino acid sequence capable of forming a matrix at body temperature is an amino acid sequence of silk, elastin, collagen, keratin, or mimic thereof, or an amino acid sequence disclosed in U.S. Pat. No. 6,355,776, which is hereby incorporated by reference.

In certain embodiments, the amino acid sequence is an Elastin-Like-Peptide (ELP) sequence. The ELP sequence includes or consists of structural peptide units or sequences that are related to, or mimics of, the elastin protein. The ELP sequence is constructed from structural units of from three to about twenty amino acids, or in some embodiments, from about four to about ten amino acids, such as about four, about five or about six amino acids. The length of the individual structural units may vary or may be uniform. Exemplary structural units are defined by SEQ ID NOS: 1-13 (below), which may be employed as repeating structural units, including tandem-repeating units, or may be employed in some combination. Thus, the ELP may comprise or consist essentially of structural unit(s) selected from SEQ ID NOS: 1-13, as defined below.

In some embodiments, including embodiments in which the structural units are ELP units, the amino acid sequence includes or consists essentially of from about 1 to about 500 structural units, or in certain embodiments about 9 to about 200 structural units, or in certain embodiments about 10 to 200 structural units, or in certain embodiments about 50 to about 200 structural units, or in certain embodiments from about 80 to about 200 structural units, or from about 80 to about 150 structural units. In some embodiments, the structural units are ELP units defined by one or more of SEQ ID NOs: 1-13. In some embodiments, the ELP includes a combination of units defined by SEQ ID NOS: 1-13. Thus, the structural units collectively may have a length of from about 50 to about 2000 amino acid residues, or from about 100 to about 800 amino acid residues, or from about 200 to about 700 amino acid residues, or from about 400 to about 600 amino acid residues. In exemplary embodiments, the amino acid sequence of the ELP structural unit includes or consists essentially of about 3 structural units, of about 7 structural units, of about 9 structural units, of about 10 structural units, of about 15 structural units, of about 20 structural units, of about 40 structural units, of about 80 structural units, of about 90 structural units, of about 100 structural units, of about 120 structural units, of about 140 structural units, about 144 structural units, of about 160 structural units, of about 180 structural units, of about 200 structural units, or of about 500 structural units. In exemplary embodiments, the structural units collectively have a length of about 45 amino acid residues, of about 90 amino acid residues, of about 100 amino acid residues, of about 200 amino acid residues, of about 300 amino acid residues, of about 400 amino acid residues, of about 500 amino acid residues, of about 600 amino acid residues, of about 700 amino acid residues, of about 720 amino acid residues, of about 800 amino acid residues, or of about 1000 amino acid residues.

The amino acid sequence may exhibit a visible and reversible inverse phase transition with the selected formulation. That is, the amino acid sequence may be structurally disordered and highly soluble in the formulation below a transition temperature (Tt), but exhibit a sharp (2-3° C. range) disorder-to-order phase transition, or coacervation, when the temperature of the formulation is raised above the Tt. In addition to temperature, length of the amino acid polymer, amino acid composition, ionic strength, pH, pressure, selected solvents, presence of organic solutes, and protein concentration may also affect the transition properties, and these may be tailored in the formulation for the desired absorption profile. Absorption profile can be easily tested by determining plasma concentration or activity of the active agent over time.

In certain embodiments, the ELP component(s) may be formed of structural units, including but not limited to:
(a) the tetrapeptide Val-Pro-Gly-Gly, or VPGG (SEQ ID NO: 1);
(b) the tetrapeptide Ile-Pro-Gly-Gly, or IPGG (SEQ ID NO: 2);
(c) the pentapeptide Val-Pro-Gly-X-Gly (SEQ ID NO: 3), or VPGXG, where X is any natural or non-natural amino acid residue except proline, and where X optionally varies among polymeric or oligomeric repeats;
(d) the pentapeptide Ala-Val-Gly-Val-Pro, or AVGVP (SEQ ID NO: 4);
(e) the pentapeptide Ile-Pro-Gly-X-Gly, or IPGXG (SEQ ID NO: 5), where X is any natural or non-natural amino acid residue, and where X optionally varies among polymeric or oligomeric repeats;
(e) the pentapeptide Ile-Pro-Gly-Val-Gly, or IPGVG (SEQ ID NO: 6);
(f) the pentapeptide Leu-Pro-Gly-X-Gly, or LPGXG (SEQ ID NO: 7), where X is any natural or non-natural amino acid residue, and where X optionally varies among polymeric or oligomeric repeats;
(g) the pentapeptide Leu-Pro-Gly-Val-Gly, or LPGVG (SEQ ID NO: 8);
(h) the hexapeptide Val-Ala-Pro-Gly-Val-Gly, or VAPGVG (SEQ ID NO: 9);
(i) the octapeptide Gly-Val-Gly-Val-Pro-Gly-Val-Gly, or GVGVPGVG (SEQ ID NO: 10);
(j) the nonapeptide Val-Pro-Gly-Phe-Gly-Val-Gly-Ala-Gly, or VPGFGVGAG (SEQ ID NO: 11);
(k) the nonapeptides Val-Pro-Gly-Val-Gly-Val-Pro-Gly-Gly, or VPGVGVPGG (SEQ ID NO: 12); and
(l) the pentapeptide Xaa-Pro-Gly-Val-Gly, or XPGVG (SEQ ID NO:13) where X is any natural or non-natural amino acid residue, and where X optionally varies among polymeric or oligomeric repeats.

Such structural units defined by SEQ ID NOS: 1-13 may form structural repeating units, or may be used in combination to form an ELP. In some embodiments, the ELP component is formed entirely (or almost entirely) of one or a combination of (e.g., 2, 3, 4, 5, 6, 7, 8, 9, or 10) structural units selected from SEQ ID NOS: 1-13. In other embodiments, at least about 75%, or at least about 80%, or at least about 90% of the ELP component is formed from one or a combination of structural units selected from SEQ ID NOS: 1-13, and which may be present as repeating units.

In certain embodiments, the ELP contains repeat units, including tandem repeating units, of Val-Pro-Gly-X-Gly (SEQ ID NO: 3), where X is as defined above, and where the percentage of Val-Pro-Gly-X-Gly (SEQ ID NO: 3) units taken with respect to the entire ELP component (which may comprise structural units other than VPGXG (SEQ ID NO: 3)) is greater than about 50%, or greater than about 75%, or greater than about 85%, or greater than about 95% of the ELP. The ELP may contain motifs of 5 to 15 structural units (e.g. about 9 or about 10 structural units) of SEQ ID NO: 3, with the guest residue X varying among at least 2 or at least 3 of the units in the motif. The guest residues may be independently selected, such as from non-polar or hydrophobic residues, such as the amino acids V, I, L, A, G, and W (and may be selected so as to retain a desired inverse phase transition property). In certain embodiments, the guest residues are selected from V, G, and A.

In certain embodiments, the ELP contains repeat units, including tandem repeating units, of Xaa-Pro-Gly-Val-Gly (SEQ ID NO: 13), where X is as defined above, and where the percentage of Xaa-Pro-Gly-Val-Gly (SEQ ID NO: 13) units taken with respect to the entire ELP component (which may comprise structural units other than XPGVG (SEQ ID NO: 13)) is greater than about 50%, or greater than about 75%, or greater than about 85%, or greater than about 95% of the ELP. The ELP may contain motifs of 5 to 15 structural units (e.g. about 9 or about 10 structural units) of SEQ ID NO: 13, with the guest residue X varying among at least 2 or at least 3 of the units in the motif. The guest residues may be independently selected, such as from non-polar or hydrophobic residues, such as the amino acids V, I, L, A, G, and W (and may be selected so as to retain a desired inverse phase transition property). In certain embodiments, the guest residues are selected from V and A.

In certain embodiments, the ELP contains repeating units, including tandem repeating units of any of SEQ ID NOs: 1-13 either alone or in combination. In some embodiments, the ELP contains repeats of two or more of any of SEQ ID NOs: 1-13 in combination. In certain embodiments, the ELP contains repeats of SEQ ID NO: 3 and SEQ ID NO: 13. In some embodiments, the ELP contains repeats of SEQ ID NO: 3 and SEQ ID NO: 13, wherein the guest residues are independently selected, such as from non-polar or hydrophobic residues, such as the amino acids V, I, L, A, G, and W (and may be selected so as to retain a desired inverse phase transition property). In certain embodiments, the guest residues are selected from V, G, and A.

In some embodiments, the ELP includes 9-mers including nine copies of one or more ELP structural units disclosed herein. In some embodiments, the ELP includes 9-mers including nine copies of a pentapeptide disclosed herein. In some embodiments, the ELP includes 9-mers including SEQ ID NOs: 3 and 13 in any combination. In some embodiments, the ELP includes a sequence alternating between SEQ ID NOs: 3 and 13. ELPs of varying numbers of 9-mers can be combined to produce ELPs with, for instance, 18, 27, 36, 45, 54, 63, 72, 81, 90, 99, 108, 117, 126, 135, 144, 153, 162, 171, or 180 copies of the 9-mer.

In certain embodiments, the ELP includes 9-mers including SEQ ID NO: 3, wherein the guest residue is selected from V, G, and A. In certain embodiments, the ELP includes 9-mers including SEQ ID NO: 3, wherein V, G, and A are in the ratio of 7:2:0 (alpha). In certain embodiments, the ELP includes 9-mers including SEQ ID NO:3, wherein V, G, and A are in the ratio of 7:0:2 (beta v1). In certain embodiments, the ELP includes 9-mers including SEQ ID NO:3, wherein V, G, and A are in the ratio of 6:0:3 (beta v2). In certain embodiments, the ELP includes 9-mers including SEQ ID NO:3, wherein V, G, and A are in the ratio of 5:2:2 (gamma). In certain embodiments, the ELP includes 9-mers including SEQ ID NO: 13, wherein the guest residue is selected from V, G, and A. In certain embodiments, the ELP includes 9-mers including SEQ ID NO:13, wherein V, G, and A are in the ratio of 5:0:4 (delta). Exemplary 9-mers are disclosed in Table 1. Table 2 demonstrates the transition temperatures of several exemplary 9-mers.

TABLE 1
Guest residue ratios in exemplary 9-mers. The ELP
polymers have hydrophobicities between the
10-mer ELP 1 series (least hydrophobic)
and the 10-mer ELP 4 series (most hydrophobic).

| ELP series | Pentamer motif | Guest residue ratio |
|---|---|---|
| 1 series | VPG [symbol] G | 5 Val:3 Gly:2 Ala |
| Alpha | VPG [symbol] G | 7 Val:2 Gly:0 Ala |
| beta v1 | VPG [symbol] G | 7 Val:0 Gly:2 Ala |
| beta v2 | VPG [symbol] G | 6 Val:0 Gly:3 Ala |
| Gamma | VPG [symbol] G | 5 Val:2 Gly:2 Ala |
| Delta | [symbol] PGVG | 5 Val:0 Gly:4 Ala |
| | VPG [symbol] G | 6 Val:3 Gly:0 Ala |
| | VPG [symbol] G | 6 Val:2 Gly:1 Ala |
| | VPG [symbol] G | 6 Val:1 Gly:2 Ala |
| | VPG [symbol] G | 6 Val:0 Gly:3 Ala |
| | VPG [symbol] G | 7 Val:1 Gly:1 Ala |
| | VPG [symbol] G | 8 Val:0 Gly:1 Ala |
| | VPG [symbol] G | 8 Val:1 Gly:0 Ala |
| 4 series | VPG [symbol] G | 10 Val:0 Gly:0 Ala |

TABLE 2
Comparison of measured transition temperatures of exemplary
9-mers to ELP1 series. The inflection of turbidity
measured using a Cary spectrophotometer is the
result of the ELP biopolymer phase transitioning.

| ELP series (10 mg/ml) | Transition temp |
|---|---|
| 1 series (pPB1023) | 37° C. |
| alpha (pPE0253) | 29° C. |
| beta v1 (pPE0254) | 28° C. |
| beta v2 (pPE0311) | 31° C. |
| gamma (pPE0255) | 29° C. |
| delta (pPE0256) | 35° C. |
| 4 series (pPE0002) | 26° C. |

In some embodiments, the ELP includes combinations of the 9-mers listed in Table 1. In some embodiments, the ELP includes combinations of the alpha, beta v1, beta v2, and/or delta 9-mers. For example, the gamma ELP is constructed by alternating between an alpha 9-mer and a beta v1 9-mer for 16 copies until a 144mer is constructed. In certain embodiments, the ELP includes combinations of alpha and beta v1 9-mers. In certain embodiments, the ELP includes combinations of alpha and beta v2 9-mers. In certain embodiments, the ELP includes combinations of alpha and delta 9-mers. In certain embodiments, the ELP includes combinations of beta v1 and beta v2 9-mers. In certain embodiments, the ELP includes combinations of beta v1 and delta 9-mers. In certain embodiments, the ELP includes combinations of beta v2 and delta 9-mers. In certain embodiments, the ELP includes combinations of alpha, beta v1, and beta v2 9-mers. In certain embodiments, the ELP includes combinations of alpha, beta v1, and delta 9-mers. In certain embodiments, the ELP includes combinations of alpha, beta v2, and delta 9-mers. For example, in particular arrangements, the ELPbeta v2 may include the following guest residues in structural units iterated in the following sequence: A-V-A-V-V-A-V-A-V. The iterated sequence may be repeated sequentially in the ELP about 10 times, about 12 times, about 15 times, about 16 times, about 20 times, about 25 times, about 30 times, or about 35 times or more. In some aspects, the ELP contains about 10 to about 20 iterated sequences. In other aspects, the ELP contains about 15 to 20 iterated sequences. In some aspects, the ELP contains about 16 iterated sequences.

In some embodiments, the ELP includes 10-mers including ten copies of one or more ELP structural units disclosed herein. In some embodiments, the ELP includes 10-mers including ten copies of a pentapeptide disclosed herein. In some embodiments, the ELP includes 10-mers including SEQ ID NOs: 3 and 13 in any combination. In some embodiments, the ELP includes a sequence alternating between SEQ ID NOs: 3 and 13. ELPs of varying numbers of 10-mers can be combined to produce ELPs with, for instance, 20, 30, 40, 60, 90, 100, 120, 150, 160, or 200 copies of the 10-mer. Exemplary 10-mers are disclosed in Table 3.

TABLE 3
Guest residue ratios in exemplary 10-mers. The ELP polymers
have hydrophobicities between the ELP 1 series (least
hydrophobic) and the ELP 4 series (most hydrophobic).

| ELP series | Pentamer motif | Guest residue ratio |
|---|---|---|
| 1 series | VPG [symbol] G | 5 Val:3 Gly:2 Ala |
| | VPG [symbol] G | 5 Val:4 Gly:1 Ala |
| | VPG [symbol] G | 5 Val:5 Gly:0 Ala |
| | VPG [symbol] G | 5 Val:2 Gly:3 Ala |
| | VPG [symbol] G | 5 Val:1 Gly:4 Ala |
| | VPG [symbol] G | 5 Val:0 Gly:5 Ala |
| | VPG [symbol] G | 6 Val:4 Gly:0 Ala |
| | VPG [symbol] G | 6 Val:3 Gly:1 Ala |
| | VPG [symbol] G | 6 Val:2 Gly:2 Ala |
| | VPG [symbol] G | 6 Val:1 Gly:3 Ala |
| | VPG [symbol] G | 6 Val:0 Gly:4 Ala |
| | VPG [symbol] G | 7 Val:3 Gly:0 Ala |
| | VPG [symbol] G | 7 Val:2 Gly:1 Ala |
| | VPG [symbol] G | 7 Val:1 Gly:2 Ala |
| | VPG [symbol] G | 7 Val:0 Gly:3 Ala |
| | VPG [symbol] G | 8 Val:2 Gly:0 Ala |
| | VPG [symbol] G | 8 Val:0 Gly:2 Ala |
| | VPG [symbol] G | 8 Val:1 Gly:1 Ala |
| | VPG [symbol] G | 9 Val:1 Gly:1 Ala |
| | VPG [symbol] G | 9 Val:0 Gly:1 Ala |
| 4 series | VPG [symbol] G | 10 Val:0 Gly:0 Ala |

In some embodiments, the ELP may form a β-turn structure. Exemplary peptide sequences suitable for creating a β-turn structure are described in International Patent Application PCT/US96/05186, which is hereby incorporated by reference in its entirety. For example, the fourth residue (X) in the sequence VPGXG (SEQ ID NO: 3), can be varied without eliminating the formation of a β-turn.

The structure of exemplary ELPs may be described using the notation ELPₖ [XᵢYⱼn], where k designates a particular ELP repeat unit, the bracketed capital letters are single letter amino acid codes, and their corresponding subscripts designate the relative ratio of each guest residue X in the structural units (where applicable), and n describes the total length of the ELP in number of the structural repeats. For example, ELP1 [V5A2G3-10] designates an ELP component containing 10 repeating units of the pentapeptide VPGXG (SEQ ID NO: 3), where X is valine, alanine, and glycine at a relative ratio of about 5:2:3; ELP1 [K₁V₂F₁-4] designates an ELP component containing 4 repeating units of the pentapeptide VPGXG (SEQ ID NO: 3), where X is lysine, valine, and phenylalanine at a relative ratio of about 1:2:1; ELP1 [K₁V₇F₁-9] designates a polypeptide containing 9 repeating units of the pentapeptide VPGXG (SEQ ID NO: 3), where X is lysine, valine, and phenylalanine at a relative ratio of about 1:7:1; ELP1 [V-5] designates a polypeptide containing 5 repeating units of the pentapeptide VPGXG (SEQ ID NO:3), where X is valine; ELP1 [V-20] designates a polypeptide containing 20 repeating units of the pentapeptide VPGXG (SEQ ID NO: 3), where X is valine; ELP2 [5] designates a polypeptide containing 5 repeating units of the pentapeptide AVGVP (SEQ ID NO: 4); ELP3 [V-5] designates a polypeptide containing 5 repeating units of the pentapeptide IPGXG (SEQ ID NO: 5), where X is valine; ELP4 [V-5] designates a polypeptide containing 5 repeating units of the pentapeptide LPGXG (SEQ ID NO: 7), where X is valine.

With respect to ELP, the Tt is a function of the hydrophobicity of the guest residue. Thus, by varying the identity of the guest residue(s) and their mole fraction(s), ELPs can be synthesized that exhibit an inverse phase transition over a broad range of temperatures. Thus, the Tt at a given ELP length may be decreased by incorporating a larger fraction of hydrophobic guest residues in the ELP sequence. Examples of suitable hydrophobic guest residues include valine, leucine, isoleucine, phenylalanine, tryptophan and methionine. Tyrosine, which is moderately hydrophobic, may also be used. Conversely, the Tt may be increased by incorporating residues, such as those selected from: glutamic acid, cysteine, lysine, aspartate, alanine, asparagine, serine, threonine, glycine, arginine, and glutamine.

For polypeptides having a molecular weight >100,000, the hydrophobicity scale disclosed in PCT/US96/05186 (which is hereby incorporated by reference in its entirety) provides one means for predicting the approximate Tt of a specific ELP sequence. For polypeptides having a molecular weight <100,000, the Tt may be predicted or determined by the following quadratic function: Tt=M0+M1X+M2X2 where X is the MW of the fusion protein, and M0=116.21; M1=−1.7499; M2=0.010349.

The ELP in some embodiments is selected or designed to provide a Tt ranging from about 10 to about 37° C., such as from about 20 to about 37° C., or from about 25° C. to about 37° C. In some embodiments, the transition temperature at physiological conditions (e.g., 0.9% saline) is from about 34° C. to 36° C., to take into account a slightly lower peripheral body temperature.

In certain embodiments, the ELP includes [VPGXG]ₘ, where m is any number from 1 to 200. In certain embodiments, the ELP includes [VPGXG]ₘ, where m is any number from 1 to 200, and each X is selected from V, G, and A. In certain embodiments, the ELP includes [VPGXG]ₘ, where m is any number from 1 to 200, each X is selected from V, G, and A, and wherein the ratio of V:G:A may be about 5:3:2. In certain embodiments, the ELP includes [VPGXG]₆₀, where each X is selected from V, G, and A, and wherein the ratio of V:G:A may be about 5:3:2. In certain embodiments, the ELP includes [VPGXG]₉₀, where each X is selected from V, G, and A, and wherein the ratio of V:G:A may be about 5:3:2. For example, the amino acid sequence capable of forming the hydrogen-bonded matrix at body temperature includes [VPGXG]₁₂₀, where each X is selected from V, G, and A, and wherein the ratio of V:G:A may be about 5:3:2. As shown herein, 120 structural units of this ELP can provide a transition temperature at about 37° C. with about 5 to 15 mg/ml (e.g., about 10 mg/ml) of protein. At concentrations of about 50 to about 100 mg/mL the phase transition temperature is about 35.5 degrees centigrade (just below body temperature), which allows for peripheral body temperature to be just less than 37° C. In some embodiments, the ELP may include [VPGXG]₁₄₄, where each X is selected from V, G, and A, and wherein the ratio of V:G:A may be about 5:3:2. In some embodiments, the ELP includes [VPGXG]₁₈₀, where each X is selected from V, G, and A, and wherein the ratio of V:G:A may be about 5:3:2.

In certain embodiments, the ELP includes [VPGXG]ₘ, where m is any number from 1 to 200, where each X is selected from V, G, and A, and wherein the ratio of V:G:A is about 7:2:0. In certain embodiments, the ELP includes [VPGXG]₆₀, where each X is selected from V, G, and A, and wherein the ratio of V:G:A is about 7:2:0. In certain embodiments, the ELP includes [VPGXG]₉₀, where each X is selected from V, G, and A, and wherein the ratio of V:G:A is about 7:2:0. In certain embodiments, the ELP includes [VPGXG]₁₀₈, where each X is selected from V, G, and A, and wherein the ratio of V:G:A is about 7:2:0. In certain embodiments, the ELP includes [VPGXG]₁₂₀, where each X is selected from V, G, and A, and wherein the ratio of V:G:A is about 7:2:0. In certain embodiments, the ELP includes [VPGXG]₁₄₄, where each X is selected from V, G, and A, and wherein the ratio of V:G:A is about 7:2:0. In certain embodiments, the ELP includes [VPGXG]₁₈₀, where each X is selected from V, G, and A, and wherein the ratio of V:G:A is about 7:2:0.

In certain embodiments, the ELP includes [VPGXG]ₘ, where m is any number from 1 to 200, where each X is selected from V, G, and A, and wherein the ratio of V:G:A is about 7:0:2. In certain embodiments, the ELP includes [VPGXG]₆₀, where each X is selected from V, G, and A, and wherein the ratio of V:G:A is about 7:0:2. In certain embodiments, the ELP includes [VPGXG]₉₀, where each X is selected from V, G, and A, and wherein the ratio of V:G:A is about 7:0:2. In certain embodiments, the ELP includes [VPGXG]₁₀₈, where each X is selected from V, G, and A, and wherein the ratio of V:G:A is about 7:0:2. In certain embodiments, the ELP includes [VPGXG]₁₂₀, where each X is selected from V, G, and A, and wherein the ratio of V:G:A is about 7:0:2. In certain embodiments, the ELP includes [VPGXG]₁₄₄, where each X is selected from V, G, and A, and wherein the ratio of V:G:A is about 7:0:2. In certain embodiments, the ELP includes [VPGXG]₁₈₀, where each X is selected from V, G, and A, and wherein the ratio of V:G:A is about 7:0:2.

In certain embodiments, the ELP includes [VPGXG]ₘ, where m is any number from 1 to 200, where each X is selected from V, G, and A, and wherein the ratio of V:G:A is about 6:0:3. In certain embodiments, the ELP includes [VPGXG]₆₀, where each X is selected from V, G, and A, and wherein the ratio of V:G:A is about 6:0:3. In certain embodiments, the ELP includes [VPGXG]₉₀, where each X is selected from V, G, and A, and wherein the ratio of V:G:A is about 6:0:3. In certain embodiments, the ELP includes [VPGXG]₁₀₈, where each X is selected from V, G, and A, and wherein the ratio of V:G:A is about 6:0:3. In certain embodiments, the ELP includes [VPGXG]₁₂₀, where each X is selected from V, G, and A, and wherein the ratio of V:G:A is about 6:0:3. In certain embodiments, the ELP includes [VPGXG]₁₄₄, where each X is selected from V, G, and A, and wherein the ratio of V:G:A is about 6:0:3. In certain embodiments, the ELP includes [VPGXG]₁₈₀, where each X is selected from V, G, and A, and wherein the ratio of V:G:A is about 6:0:3.

In certain embodiments, the ELP includes [VPGXG]ₘ, where m is any number from 1 to 200, where each X is selected from V, G, and A, and wherein the ratio of V:G:A is about 5:2:2. In certain embodiments, the ELP includes [VPGXG]₆₀, where each X is selected from V, G, and A, and wherein the ratio of V:G:A is about 5:2:2. In certain embodiments, the ELP includes [VPGXG]₉₀, where each X is selected from V, G, and A, and wherein the ratio of V:G:A is about 5:2:2. In certain embodiments, the ELP includes [VPGXG]₁₀₈, where each X is selected from V, G, and A, and wherein the ratio of V:G:A is about 5:2:2. In certain embodiments, the ELP includes [VPGXG]₁₂₀, where each X is selected from V, G, and A, and wherein the ratio of V:G:A is about 5:2:2. In certain embodiments, the ELP includes [VPGXG]₁₄₄, where each X is selected from V, G, and A, and wherein the ratio of V:G:A is about 5:2:2. In certain embodiments, the ELP includes [VPGXG]₁₈₀, where each X is selected from V, G, and A, and wherein the ratio of V:G:A is about 5:2:2.

In certain embodiments, the ELP includes [VPGXG]ₘ, where m is any number from 1 to 200, where each X is selected from V, G, and A, and wherein the ratio of V:G:A is about 10:0:0. In certain embodiments, the ELP includes [VPGXG]₆₀, where each X is selected from V, G, and A, and wherein the ratio of V:G:A is about 10:0:0. In certain embodiments, the ELP includes [VPGXG]₉₀, where each X is selected from V, G, and A, and wherein the ratio of V:G:A is about 10:0:0. In certain embodiments, the ELP includes [VPGXG]₁₀₈, where each X is selected from V, G, and A, and wherein the ratio of V:G:A is about 10:0:0. In certain embodiments, the ELP includes [VPGXG]₁₂₀, where each X is selected from V, G, and A, and wherein the ratio of V:G:A is about 10:0:0. In certain embodiments, the ELP includes [VPGXG]₁₄₄, where each X is selected from V, G, and A, and wherein the ratio of V:G:A is about 10:0:0. In certain embodiments, the ELP includes [VPGXG]₁₈₀, where each X is selected from V, G, and A, and wherein the ratio of V:G:A is about 10:0:0.

In certain embodiments, the ELP includes [VPGXG]ₘ, where m is any number from 1 to 100, where each X is selected from V and L, and wherein the ratio of V:L is about 3:7 or about 4:6 or about 1:1 or about 6:4 or about 3:7. In certain embodiments, the ELP includes [VPGXG]₆₀, where each X is selected from V and L, and wherein the ratio of V:L is about 3:7 or about 4:6 or about 1:1 or about 6:4 or about 3:7. In certain embodiments, the ELP includes [VPGXG]₅₀, where each X is selected from V and L, and wherein the ratio of V:L is about 3:7 or about 4:6 or about 1:1 or about 6:4 or about 3:7. In certain embodiments, the ELP includes [VPGXG]₄₀, where each X is selected from V and L, and wherein the ratio of V:L is about 3:7 or about 4:6 or about 1:1 or about 6:4 or about 3:7. In certain embodiments, the ELP includes [VPGXG]₃₀, where each X is selected from V and L, and wherein the ratio of V:L is about 3:7 or about 4:6 or about 1:1 or about 6:4 or about 3:7 In certain embodiments, the ELP includes [VPGXG]₂₀, where each X is selected from V and L, and wherein the ratio of V:L is about 3:7 or about 4:6 or about 1:1 or about 6:4 or about 3:7.

In certain embodiments, the ELP includes [XPGVG]ₘ, where m is any number from 1 to 200. In certain embodiments, the ELP includes [XPGVG]ₘ, where m is any number from 1 to 200, and each X is selected from V, G, and A. In certain embodiments, the ELP includes [XPGVG]ₘ, where m is any number from 1 to 200, each X is selected from V, G, and A and wherein the ratio of V:G:A is about 5:0:4. In certain embodiments, the ELP includes [XPGVG]₆₀, where each X is selected from V, G, and A, and wherein the ratio of V:G:A is about 5:0:4. In certain embodiments, the ELP includes [XPGVG]₉₀, where each X is selected from V, G, and A, and wherein the ratio of V:G:A is about 5:0:4. In certain embodiments, the ELP includes [XPGVG]₁₂₀, where each X is selected from V, G, and A, and wherein the ratio of V:G:A is about 5:0:4. In certain embodiments, the ELP includes [XPGVG]₁₄₄, where each X is selected from V, G, and A, and wherein the ratio of V:G:A is about 5:0:4. In certain embodiments, the ELP includes [XPGVG]₁₈₀, where each X is selected from V, G, and A, and wherein the ratio of V:G:A is about 5:0:4.

In certain embodiments, the ELP includes [VPGVG]ₘ where m is any number from 1 to 200. In some embodiments, the ELP includes [VPGVG]₆₀, [VPGVG]₉₀, or [VPGVG]₁₂₀. As shown herein, 120 structural units of this ELP can provide a transition temperature at about 37° C. with about 0.005 to about 0.05 mg/ml (e.g., about 0.01 mg/ml) of protein. Alternatively, the ELP includes [VPGXG]₁₄₄ or [XPGVG]₁₄₄. As shown herein (Table 2), 144 structural units of either of these ELPs can provide a transition temperature at 28° C. to 35° C. inclusive.

In various embodiments, the intended subject is human, and the body temperature is about 37° C., and thus the therapeutic agent is designed to provide a sustained release at or near this temperature (e.g. between about 28° C. to about 37° C.). A slow release into the circulation with reversal of hydrogen bonding and/or hydrophobic interactions is driven by a drop in concentration as the product diffuses at the injection site, even though body temperature remains constant. In other embodiments, the subject is a non-human mammal, and the therapeutic agent is designed to exhibit a sustained release at the body temperature of the mammal, which may be from about 30 to about 40° C. in some embodiments, such as for certain domesticated pets (e.g., dog or cat) or livestock (e.g., cow, horse, sheep, or pig). Generally, the Tt is higher than the storage conditions of the formulation (which may be from 2 to about 25° C., or from 15 to 22° C.), such that the therapeutic agent remains in solution for injection.

In some embodiments, the ELP can provide a transition temperature at a range of 27° C. to 36° C. inclusive. In some embodiments, the ELP can provide a transition temperature at a range of 28° C. to 35° C. inclusive. In some embodiments, the ELP can provide a transition temperature at a range of 29° C. to 34° C. inclusive. In some embodiments, the ELP can provide a transition temperature at a range of 27° C. to 33° C. inclusive. In some embodiments, the ELP can provide a transition temperature at a range of 30° C. to 33° C. inclusive. In some embodiments, the ELP can provide a transition temperature at a range of 31° C. to 31° C. inclusive. In some embodiments, the ELP can provide a transition temperature of 27° C., 28° C., 29° C., 30° C., 31° C., 32° C., 33° C., 34° C., 35° C., or 36° C. In some embodiments, the ELP can provide a transition temperature at a range of 28° C. to 35° C. inclusive at a protein concentration of 10 mg/mL in 110 mM NaCl.

Elastin-like-peptide (ELP) protein polymers and recombinant fusion proteins can be prepared as described in U.S. Patent Publication No. 2010/0022455, which is hereby incorporated by reference. In some embodiments, the ELP protein polymers are constructed through recursive ligation to rapidly clone DNA encoding highly repetitive polypeptides of any sequence and specified length over a large range of molecular weights. In a single cycle, two halves of a parent plasmid, each containing a copy of an oligomer, are ligated together, thereby dimerizing the oligomer and reconstituting a functional plasmid. This process is carried out recursively to assemble an oligomeric gene with the desired number of repeats. For example, one ELP structural subunit (e.g. a pentapeptide or a 9-mer of pentapeptides) is inserted into a vector. The vector is digested, and another ELP structural unit (e.g. a pentapeptide or a 9-mer of pentapeptides) is inserted. Each subsequent round of digestion and ligation doubles the number of ELP structural units contained in the resulting vector until the ELP polymer is the desired length. By varying the number of pentapeptides in the initial structural unit, ELPs of varying length can easily be constructed. Alternative means of construction (i.e. other than recursive ligation) can be used to produce alternative lengths of ELP.

In some embodiments, the vector contains one or more additional amino acids or ELP structural unit repeats. For example, pPE0248 adds an additional pentamer repeat to the N terminus of the 144mer with valine in the guest position and an additional pentamer to the C terminus with a tryptophan in the guest residue position. The tryptophan may be used as a means to increase the extinction coefficient of the molecule, allowing for better measurement of absorbance, for instance at 280 nm, which can be useful for determination of protein concentration, or for monitoring protein content during purification. The pentamers added to either end can also be designed so as the encoding DNA contains restriction enzyme recognition sites for cloning of fusion partners on to either end of the ELP coding sequence.

In some embodiments, the therapeutic agent includes an active agent and one or more ELPs. In some embodiments, the therapeutic agent includes an active agent with one or more ELPs at either the N- or C-terminus. In some embodiments, the therapeutic agent includes an active agent with one or more ELPs at both the N- or C-termini. In some embodiments, the ELPs are approximately the same size. In some embodiments, the ELPs differ in size. In some embodiments, an ELP at one terminus is larger than an ELP at the other terminus. In some embodiments, an ELP at the N-terminus is larger than an ELP at the C-terminus. In some embodiments, an ELP at the C-terminus is larger than an ELP at the N-terminus.

### Methods of Treatment

In other aspects, the present disclosure provides methods for treating or preventing diseases characterized by abnormalities in bone resorption and formation. In some embodiments, the disease is osteoporosis. In some embodiments, the disease is hypoparathyroidism. Generally, the patient may be a human or non-human animal patient (e.g., dog, cat, cow, or horse). Preferably, the patient is human.

Hypoparathyroidism is a rare disorder characterized by hypocalcemia and low or insufficient PTH concentrations. The acute clinical manifestations of hypoparathyroidism are related to hypocalcemia and can include life-threatening arrhythmias, laryngospasm and seizures. Chronic manifestations of the disease include hyperphosphatemia, hypercalciuria, nephrolithiasis and nephrocalcinosis, abnormal skeletal remodeling, neurocognitive complaints and reduced quality of life. The renal manifestations may be due to the treatment of hypoparathyroidism with calcium and active vitamin D but not directly a feature of the disease itself.

Osteoporosis is a systemic disorder characterized as the depletion of bone mass with structural deterioration of bone tissue. This results in a decrease in bone mineral density (BMD) and a predisposition to fragility fractures.

In some embodiments, the compositions of the present disclosure are administered to treat, ameliorate, decrease symptoms, or delay progression of a disease. In some embodiments, the compositions of the present disclosure are administered until a disease is treated, ameliorated, the symptoms are decreased, or the progression is delayed. In some embodiments, the compositions of the present disclosure are administered for about one day, about one week, about one month, about six months, about one year, about years or more. In some embodiments, the compositions of the present disclosure are administered for longer than two years. In some embodiments, the compositions of the present disclosure are administered continuously or regularly. In some embodiments, the compositions of the present disclosure are administered intermittently.

In some embodiments, administration of the compositions of the present disclosure increase bone formation in a treated subject compared to untreated patients, or the same patient before treatment. In some embodiments, administration of the compositions of the present disclosure increase bone formation about 1% to about 100% in a treated subject compared to untreated patients, or the same patient before treatment. In some embodiments, administration of the compositions of the present disclosure increase bone formation by about 1%, about 2%, about 3%, about 4%, about 5%, about 6%, about 7%, about 8%, about 9%, about 10%, about 15%, about 20%, about 30%, about 40%, about 50%, about 60%, about 70%, about 80%, about 90%, about 95%, about 96%, about 97%, about 98%, about 99%, or about 100% in a treated subject compared to untreated patients, or the same patient before treatment.

In some embodiments, administration of the compositions of the present disclosure increases bone formation in a treated patient at about 1 day to about 5 years compared to an untreated patient or the same patient before treatment. In some embodiments, administration of the compositions of the present disclosure increases bone formation in a treated subject at about 1 day, about 2 days, about 3 days, about 4 days, about 5 days, about 6 days, about 7 days, about 1 week, about 2 weeks, about 3 weeks, about 4 weeks, about one month, about two months, about three months, about four months, about five months, about six months, about seven months, about eight months, about nine months, about ten months, about 11 months, about 12 months, about 1 year, or about 2 years compared to an untreated patient or the same patient before treatment.

In some embodiments, administration of the compositions of the present disclosure increases bone formation in a treated subject by about 1%, about 2%, about 3%, about 4%, about 5%, about 6%, about 7%, about 8%, about 9%, about 10%, about 15%, about 20%, about 30%, about 40%, about 50%, about 60%, about 70%, about 80%, about 90%, about 95%, about 96%, about 97%, about 98%, about 99%, or about 100%, at about 1 day, about 2 days, about 3 days, about 4 days, about 5 days, about 6 days, about 7 days, about 1 week, about 2 weeks, about 3 weeks, about 4 weeks, about one month, about two months, about three months, about four months, about five months, about six months, about seven months, about eight months, about nine months, about ten months, about 11 months, about 12 months, about 1 year, or about 2 years compared to an untreated patient or the same patient before treatment.

In some embodiments, administration of the compositions of the present disclosure increases bone mineral density (BMD) in a treated subject compared to untreated patients, or the same patient before treatment. BMD may be measured by any appropriate means, and in some embodiments by dual energy X-ray absorptiometry (DXA). In some embodiments, administration of the compositions of the present disclosure increases BMD about 1% to about 100% in a treated subject compared to untreated patients, or the same patient before treatment. In some embodiments, administration of the compositions of the present disclosure increases BMD by about 1%, about 2%, about 3%, about 4%, about 5%, about 6%, about 7%, about 8%, about 9%, about 10%, about 15%, about 20%, about 30%, about 40%, about 50%, about 60%, about 70%, about 80%, about 90%, about 95%, about 96%, about 97%, about 98%, about 99%, or about 100% in a treated subject compared to untreated patients, or the same patient before treatment.

In some embodiments, administration of the compositions of the present disclosure increases BMD in a treated patient at about 1 day to about 5 years compared to an untreated patient or the same patient before treatment. In some embodiments, administration of the compositions of the present disclosure increases BMD in a treated subject at about 1 day, about 2 days, about 3 days, about 4 days, about 5 days, about 6 days, about 7 days, about 1 week, about 2 weeks, about 3 weeks, about 4 weeks, about one month, about two months, about three months, about four months, about five months, about six months, about seven months, about eight months, about nine months, about ten months, about 11 months, about 12 months, about 1 year, or about 2 years compared to an untreated patient or the same patient before treatment.

In some embodiments, administration of the compositions of the present disclosure increases BMD in a treated subject by about 1%, about 2%, about 3%, about 4%, about 5%, about 6%, about 7%, about 8%, about 9%, about 10%, about 15%, about 20%, about 30%, about 40%, about 50%, about 60%, about 70%, about 80%, about 90%, about 95%, about 96%, about 97%, about 98%, about 99%, or about 100%, at about 1 day, about 2 days, about 3 days, about 4 days, about 5 days, about 6 days, about 7 days, about 1 week, about 2 weeks, about 3 weeks, about 4 weeks, about one month, about two months, about three months, about four months, about five months, about six months, about seven months, about eight months, about nine months, about ten months, about 11 months, about 12 months, about 1 year, or about 2 years compared to an untreated patient or the same patient before treatment.

In some embodiments, administration of the compositions of the present disclosure decreases incidence and/or risk of bone fracture in a treated subject compared to untreated patients, or the same patient before treatment. In some embodiments, administration of the compositions of the present disclosure decreases incidence and/or risk of bone fracture about 1% to about 100% in a treated subject compared to untreated patients, or the same patient before treatment. In some embodiments, administration of the compositions of the present disclosure decreases incidence and/or risk of bone fracture by about 1%, about 2%, about 3%, about 4%, about 5%, about 6%, about 7%, about 8%, about 9%, about 10%, about 15%, about 20%, about 30%, about 40%, about 50%, about 60%, about 70%, about 80%, about 90%, about 95%, about 96%, about 97%, about 98%, about 99%, or about 100% in a treated subject compared to untreated patients, or the same patient before treatment.

In some embodiments, administration of the compositions of the present disclosure decreases incidence and/or risk of bone fracture in a treated patient at about 1 day to about 5 years compared to an untreated patient or the same patient before treatment. In some embodiments, administration of the compositions of the present disclosure decreases incidence and/or risk of bone fracture in a treated subject at about 1 day, about 2 days, about 3 days, about 4 days, about 5 days, about 6 days, about 7 days, about 1 week, about 2 weeks, about 3 weeks, about 4 weeks, about one month, about two months, about three months, about four months, about five months, about six months, about seven months, about eight months, about nine months, about ten months, about 11 months, about 12 months, about 1 year, or about 2 years compared to an untreated patient or the same patient before treatment.

In some embodiments, administration of the compositions of the present disclosure decreases incidence and/or risk of bone fracture in a treated subject by about 1%, about 2%, about 3%, about 4%, about 5%, about 6%, about 7%, about 8%, about 9%, about 10%, about 15%, about 20%, about 30%, about 40%, about 50%, about 60%, about 70%, about 80%, about 90%, about 95%, about 96%, about 97%, about 98%, about 99%, or about 100%, at about 1 day, about 2 days, about 3 days, about 4 days, about 5 days, about 6 days, about 7 days, about 1 week, about 2 weeks, about 3 weeks, about 4 weeks, about one month, about two months, about three months, about four months, about five months, about six months, about seven months, about eight months, about nine months, about ten months, about 11 months, about 12 months, about 1 year, or about 2 years compared to an untreated patient or the same patient before treatment.

In some embodiments, administration of the compositions of the present disclosure increases biomarkers of bone formation in a treated subject compared to untreated patients, or the same patient before treatment. In some embodiments, the biomarkers of bone resorption remain unchanged. In some embodiments, administration of the compositions of the present disclosure increases biomarkers of bone formation about 1% to about 100% in a treated subject compared to untreated patients, or the same patient before treatment. In some embodiments, administration of the compositions of the present disclosure increases biomarkers of bone formation by about 1%, about 2%, about 3%, about 4%, about 5%, about 6%, about 7%, about 8%, about 9%, about 10%, about 15%, about 20%, about 30%, about 40%, about 50%, about 60%, about 70%, about 80%, about 90%, about 95%, about 96%, about 97%, about 98%, about 99%, or about 100% in a treated subject compared to untreated patients, or the same patient before treatment.

In some embodiments, administration of the compositions of the present disclosure increases biomarkers of bone formation in a treated patient at about 1 day to about 5 years compared to an untreated patient or the same patient before treatment. In some embodiments, administration of the compositions of the present disclosure increases biomarkers of bone formation in a treated subject at about 1 day, about 2 days, about 3 days, about 4 days, about 5 days, about 6 days, about 7 days, about 1 week, about 2 weeks, about 3 weeks, about 4 weeks, about one month, about two months, about three months, about four months, about five months, about six months, about seven months, about eight months, about nine months, about ten months, about 11 months, about 12 months, about 1 year, or about 2 years compared to an untreated patient or the same patient before treatment.

In some embodiments, administration of the compositions of the present disclosure increases biomarkers of bone formation in a treated subject by about 1%, about 2%, about 3%, about 4%, about 5%, about 6%, about 7%, about 8%, about 9%, about 10%, about 15%, about 20%, about 30%, about 40%, about 50%, about 60%, about 70%, about 80%, about 90%, about 95%, about 96%, about 97%, about 98%, about 99%, or about 100%, at about 1 day, about 2 days, about 3 days, about 4 days, about 5 days, about 6 days, about 7 days, about 1 week, about 2 weeks, about 3 weeks, about 4 weeks, about one month, about two months, about three months, about four months, about five months, about six months, about seven months, about eight months, about nine months, about ten months, about 11 months, about 12 months, about 1 year, or about 2 years compared to an untreated patient or the same patient before treatment.

In some embodiments, administration of the compositions of the present disclosure decreases biomarkers of bone formation in a treated subject compared to untreated patients, or the same patient before treatment. In some embodiments, administration of the compositions of the present disclosure decreases biomarkers of bone formation about 1% to about 100% in a treated subject compared to untreated patients, or the same patient before treatment. In some embodiments, administration of the compositions of the present disclosure decreases biomarkers of bone formation by about 1%, about 2%, about 3%, about 4%, about 5%, about 6%, about 7%, about 8%, about 9%, about 10%, about 15%, about 20%, about 30%, about 40%, about 50%, about 60%, about 70%, about 80%, about 90%, about 95%, about 96%, about 97%, about 98%, about 99%, or about 100% in a treated subject compared to untreated patients, or the same patient before treatment.

In some embodiments, administration of the compositions of the present disclosure decreases biomarkers of bone formation in a treated patient at about 1 day to about 5 years compared to an untreated patient or the same patient before treatment. In some embodiments, administration of the compositions of the present disclosure decreases biomarkers of bone formation in a treated subject at about 1 day, about 2 days, about 3 days, about 4 days, about 5 days, about 6 days, about 7 days, about 1 week, about 2 weeks, about 3 weeks, about 4 weeks, about one month, about two months, about three months, about four months, about five months, about six months, about seven months, about eight months, about nine months, about ten months, about 11 months, about 12 months, about 1 year, or about 2 years compared to an untreated patient or the same patient before treatment.

In some embodiments, administration of the compositions of the present disclosure decreases biomarkers of bone formation in a treated subject by about 1%, about 2%, about 3%, about 4%, about 5%, about 6%, about 7%, about 8%, about 9%, about 10%, about 15%, about 20%, about 30%, about 40%, about 50%, about 60%, about 70%, about 80%, about 90%, about 95%, about 96%, about 97%, about 98%, about 99%, or about 100%, at about 1 day, about 2 days, about 3 days, about 4 days, about 5 days, about 6 days, about 7 days, about 1 week, about 2 weeks, about 3 weeks, about 4 weeks, about one month, about two months, about three months, about four months, about five months, about six months, about seven months, about eight months, about nine months, about ten months, about 11 months, about 12 months, about 1 year, or about 2 years compared to an untreated patient or the same patient before treatment.

In some embodiments, administration of the compositions of the present disclosure stabilizes serum calcium levels in a treated subject compared to untreated patients, or the same patient before treatment. In some embodiments, administration of the compositions of the present disclosure stabilizes serum calcium levels in a treated patient to about 7.5 mg/dL to about 10.6 mg/dL. In some embodiments, administration of the compositions of the present disclosure stabilizes serum calcium levels in a treated patient at about 1 day to about 5 years compared to an untreated patient or the same patient before treatment. In some embodiments, administration of the compositions of the present disclosure stabilizes serum calcium levels in a treated subject at about 1 day, about 2 days, about 3 days, about 4 days, about 5 days, about 6 days, about 7 days, about 1 week, about 2 weeks, about 3 weeks, about 4 weeks, about one month, about two months, about three months, about four months, about five months, about six months, about seven months, about eight months, about nine months, about ten months, about 11 months, about 12 months, about 1 year, or about 2 years compared to an untreated patient or the same patient before treatment.

### Formulations

The present disclosure provides sustained release formulations including a therapeutic agent disclosed herein and one or more pharmaceutically acceptable excipients and/or diluents. For example, such excipients include salts, and other excipients that may act to stabilize hydrogen bonding. Any appropriate excipient known in the art may be used. Exemplary excipients include, but are not limited to, amino acids such as histidine, glycine, or arginine; glycerol; sugars, such as sucrose; surface active agents such as polysorbate 20 and polysorbate 80; acetic acid; citric acid; sodium citrate; antioxidants; salts including alkaline earth metal salts such as sodium, potassium, and calcium; counter ions such as chloride, acetate and phosphate; sugar alcohols (e.g. mannitol); preservatives (e.g. m-cresol); sugar alcohols (e.g. mannitol, sorbitol); and buffering agents. Exemplary salts include sodium chloride, potassium chloride, magnesium chloride, calcium chloride, sodium acetate, sodium phosphate dibasic, sodium phosphate monobasic, sodium phosphate, and potassium phosphate.

The therapeutic agent is formulated at a pH, ionic strength, and generally with excipients sufficient to enable the formation of the matrix at body temperature (e.g., 37° C., or at from 34 to 36° C. in some embodiments). The therapeutic agent is generally prepared such that it does not form the matrix at storage conditions. The formulation can be stored frozen, refrigerated or at room temperature. Storage conditions are generally less than the transition temperature of the formulation, such as less than about 32° C., or less than about 30° C., or less than about 27° C., or less than about 25° C., or less than about 20° C., or less than about 15° C. For example, the formulation may be isotonic with blood or have an ionic strength that mimics physiological conditions. For example, the formulation may have an ionic strength of at least that of 25 mM Sodium Chloride, or at least that of 30 mM Sodium chloride, or at least that of 40 mM Sodium Chloride, or at least that of 50 mM Sodium Chloride, or at least that of 75 mM Sodium Chloride, or at least that of 100 mM Sodium Chloride, or at least that of 150 mM Sodium Chloride. In certain embodiments, the formulation has an ionic strength equivalent to that of 0.9% saline (154 mM sodium chloride).

In some embodiments, the formulation is stable at storage conditions. Storage conditions may be any conditions used to stably store a formulation. In some embodiments, the formulation is refrigerated. In some embodiments, the formulation is frozen. In some embodiments, the storage conditions include temperatures of less than about 30° C. In some embodiments, the storage conditions include temperatures of about 2° C. to about 8° C. In some embodiments, the storage conditions include temperatures below 0° C. In some embodiments, the storage conditions include temperatures of about −15° C. to about −80° C.

Stability can be measured using any appropriate means in the art. Generally, a stable formulation is one that shows less than a 5% increase in degradation products or impurities. In some embodiments, the formulation is stable for at least about 1 month, at least about 2 months, at least about 3 months, at least about 4 months, at least about 5 months, at least about 6 months, or at least about one year or more at the storage conditions. In some embodiments, the formulation is stable for at least about 1 month, at least about 2 months, at least about 3 months, at least about 4 months, at least about 5 months, at least about 6 months, at least about one year, or at least about two years or more at 2-8° C. In some embodiments, the formulation is stable for at least about 1 month, at least about 2 months, at least about 3 months, at least about 4 months, at least about 5 months, at least about 6 months, at least about one year, or at least about two years or more at 25° C. In some embodiments, the formulation is stable for at least about 1 month, at least about 2 months, at least about 3 months, at least about 4 months, at least about 5 months, at least about 6 months, at least about one year, or at least about two years or more at −15° C. to about −80° C.

In some embodiments, the formulation includes two or more of calcium chloride, magnesium chloride, potassium chloride, potassium phosphate monobasic, sodium chloride, sodium phosphate dibasic, sodium phosphate monobasic, histidine, arginine, glycine, glycerol, antimicrobial preservative (e.g. metacresol), tonicity-adjusting agent (e.g. mannitol), glacial acetic acid, sodium acetate trihydrate; sucrose, sodium phosphate monobasic monohydrate, sodium phosphate dibasic heptahydrate, zinc, m-cresol, phenol, sorbitol, polysorbate 80, and polysorbate 20.

In some embodiments, the formulation includes histidine or another amino acid at a range of about 10 mM to about 100 mM histidine. In some embodiments, the formulation includes histidine or another amino acid at a range of about 10 mM to about 30 mM histidine. In some embodiments, the formulation includes histidine or another amino acid at a range of about 15 mM to about 25 mM histidine. In some embodiments, the formulation includes NaCl at a range of about 10 mM to about 165 mM NaCl. In some embodiments, the formulation includes between about 50 mM and about 165 mM NaCl. In some embodiments, the formulation includes between about 54 mM and about 162 mM NaCl. In some embodiments, the formulation includes between about 110 mM and about 162 mM NaCl. In some embodiments, the formulation includes sodium phosphate at a range of about 1 mM to about 20 mM. In some embodiments, the formulation includes sodium phosphate at a range of about 5 mM to about 15 mM. In some embodiments, the formulation includes sodium phosphate monobasic at a range of about 2 mM to about 10 mM. In some embodiments, the formulation includes sodium phosphate monobasic at a range of about 4 mM to about 8 mM. In some embodiments, the formulation includes sodium phosphate dibasic at a range of about 1 mM to about 10 mM. In some embodiments, the formulation includes sodium phosphate dibasic at a range of about 2 mM to about 7 mM. In some embodiments, the formulation includes sodium phosphate dibasic at a range of about 2 mM to about 5 mM. In some embodiments, the formulation includes polysorbate 20 at a range of about 0.01% to about 0.2%. In some embodiments, the formulation includes polysorbate 80 at a range of about 0.01% to about 0.2%. In some embodiments, the formulation includes sodium phosphate, sodium chloride, sodium phosphate monobasic, sodium phosphate dibasic, and polysorbate 20. In some embodiments, the formulation includes about 10 mM sodium phosphate (about 7 mM sodium phosphate monobasic and about 3 mM sodium phosphate dibasic), about 110 mM sodium chloride, and about 0.1% polysorbate 20.

In some embodiments, the formulation is formulated at physiological pH. In some embodiments, the formulation is formulated at a pH in the range of about 5.5 to about 7.5. In some embodiments, the formulation is formulated at a pH in the range of about 6.0 to about 7.0. In some embodiments, the formulation is formulated at a pH in the range of about 6.5 to about 7.0. In some embodiments, formulations with a lower pH demonstrate improved formulation stability compared to formulations at a higher pH. In some embodiments, formulations with a pH of about 6.5 demonstrate improved stability compared to formulations with a pH of about 7.0. In some embodiments, formulations with a pH of about 6.0 demonstrate improved stability compared to formulations with a pH of about 6.5. In some embodiments, formulations with a pH of about 5.5 demonstrate improved stability compared to formulations with a pH of about 6.0. In some embodiments, formulations with a pH of about 5.0 demonstrate improved stability compared to formulations with a pH of about 5.5. In some embodiments, formulations with a pH of about 4.5 demonstrate improved stability compared to formulations with a pH of about 5.0. In some embodiments, formulations with a pH of about 4.0 demonstrate improved stability compared to formulations with a pH of about 4.5. In some embodiments, formulations with a lower pH maintain a higher percentage of monomers compared to formulations at a higher pH. In some embodiments, formulations with a pH of about 6.5 maintain a higher percentage of monomers compared to formulations with a pH of about 7.0.

In some embodiments, formulations with a pH of about 6.0 maintain a higher percentage of monomers compared to formulations with a pH of about 6.5. In some embodiments, formulations with a pH of about 6.0 maintain a lower percentage of degradation products compared to formulations with a pH of about 6.5. In some embodiments, formulations with a pH of about 5.5 maintain a lower percentage of degradation products compared to formulations with a pH of about 6.0. In some embodiments, formulations with a pH of about 5.0 maintain a lower percentage of degradation products compared to formulations with a pH of about 5.5. In some embodiments, formulations with a pH of about 4.5 maintain a lower percentage of degradation products compared to formulations with a pH of about 5.0. In some embodiments, formulations with a pH of about 4.0 maintain a lower percentage of degradation products compared to formulations with a pH of about 4.5.

In some embodiments, formulations comprise 25 mM histidine, 110 mM sodium chloride, pH 6.0. In some embodiments, formulations comprise 6.8 mM acetic acid, 1.2 mM sodium acetate, 250 mM D-mannitol, 0.3% m-cresol, pH 4.0.

The protein concentration of the therapeutic agent in the formulation is tailored to drive the formation of the matrix at the temperature of administration. For example, higher protein concentrations help drive the formation of the matrix, and the protein concentration needed for this purpose varies depending on the ELP series used. For example, in embodiments using an ELP1-120, or amino acid sequences with comparable transition temperatures, the protein is present in the range of about 1 mg/mL to about 200 mg/mL, or is present in the range of about 30 mg/mL to about 150 mg/mL. In embodiments using an ELP4-120, or amino acid sequences with comparable transition temperatures, the protein is present in the range of about 0.005 mg/mL to about 10 mg/mL, or is present in the range of about 0.01 mg/mL to about 5 mg/mL.

In some embodiments, the therapeutic agent may be present in the range of about 0.5 mg/mL to about 200 mg/mL, or is present in the range of about 30 mg/mL to about 150 mg/mL. In some embodiments, the therapeutic agent is present in the range of about 50 mg/mL to about 125 mg/mL, or the range of about 75 mg/mL to about 110 mg/mL. In some embodiments, the therapeutic agent is present at a concentration of about 100 mg/mL.

### Dosage and Administration

In some aspects, the disclosure provides a method for delivering a sustained release regimen of an active agent disclosed herein. The method includes administering the pharmaceutical composition described herein to a subject in need, wherein the pharmaceutical composition is administered from about 1 to about 8 times per month. In some embodiments, the pharmaceutical composition is administered about 1 time, about 2 times, about 3 times, and/or about 4 times per month.

In some embodiments, the pharmaceutical composition is administered weekly. In some embodiments, the pharmaceutical composition is administered daily. In some embodiments, the pharmaceutical composition is administered from one to three times weekly. In some embodiments, the pharmaceutical composition is administered once every two weeks. In some embodiments, the pharmaceutical composition is administered from one to two times a month. In particular embodiments, the pharmaceutical composition is administered about 1 time per month. In some embodiments, the pharmaceutical composition is administered about once every 2 months, about once every 3 months, about once every 4 months, about once every 5 months, and/or about once every 6 months. The pharmaceutical composition can be packaged in the form of pre-filled pens or syringes for administration once per week, twice per week, or from one to eight times per month, or alternatively filled in conventional vials and the like.

In some embodiments, the formulation is administered about monthly, and may be administered subcutaneously or via a pump. In some embodiments, the formulation is administered about weekly, and may be administered subcutaneously or via a pump. In some embodiments, the site of administration is not a pathological site, for example, is not the intended site of action.

In some embodiments, the pharmaceutical compositions disclosed herein are administered chronically. In some embodiments, the pharmaceutical compositions disclosed herein are administered for about 6 months, for about 7 months, for about 8 months, for about 9 months, for about 10 months, for about 11 months, for about 1 year, for about 2 years, for about 3 years, for about 4 years, for about 5 years, for about 10 years or more. The pharmaceutical compositions may be administered at any required dose and/or frequency disclosed herein.

In some embodiments, the pharmaceutical compositions disclosed herein are administered continuously or regularly. In some embodiments, the pharmaceutical compositions disclosed herein are administered intermittently. In some embodiments, administration of the pharmaceutical compositions of the present disclosure are halted during treatment of the patient with a different therapeutic, and then re-started after that course of treatment is concluded. In some embodiments, intermittent administration of the pharmaceutical compositions of the present disclosure has a different effect than continuous or regular administration of the same composition. For example, intermittent administration may influence which genes are turned on or off in the patient, thereby altering the effects of the pharmaceutical composition.

In some embodiments, the pharmaceutical compositions disclosed herein are administered until disease or disorder symptoms improve. In some embodiments, the pharmaceutical compositions disclosed herein are administered until disease or disorder symptoms are ameliorated, delayed, and/or cured.

In some embodiments, the pharmaceutical compositions disclosed herein are administered before the patient begins to exhibit one or more disease or disorder symptoms. In some embodiments, the pharmaceutical compositions disclosed herein are administered at the onset of disease or disorder symptoms.

The therapeutic agent is formulated generally for “systemic delivery,” meaning that the agent is not delivered locally to a pathological site or a site of action. Instead, the agent is absorbed into the bloodstream from the injection site, where the agent acts systemically or is transported to a site of action via the circulation. The therapeutic agent may be administered by any known route, such as for example, orally, intravenously, intramuscularly, nasally, subcutaneously, via injection pump, via transdermal patch, intra-vaginally, and intra-rectally. In some embodiments, the route of administration influences the efficacy of treatment. For example, administration of a PTH via a pump may be more effective at treating disease than subcutaneous administration of the same therapeutic two times a day.

In some embodiments, the formulation is generally for subcutaneous administration. In some embodiments, the pharmacokinetic (PK) parameters are prolonged when the agent is administered subcutaneously. In some embodiments, the half-life of the fusion protein is prolonged. In some embodiments, the PK parameters when the agent is administered subcutaneously are prolonged compared with the agent administered by other means (e.g. intravenously). In some embodiments, the depot of the agent is prolonged when the agent is administered subcutaneously compared with the agent administered by other means (e.g. intravenously). By providing a slow absorption from the injection site, renal clearance and degradation can be controlled, thereby achieving the desired PK profile.

Advantageously, the compositions provide for prolonged pharmacokinetic exposure due to sustained release of the active agent. In particular aspects, the maximal exposure level may be achieved at about 10 hours, about 24 hours, about 48 hours or about 72 hours after administration; typically the maximum exposure level is achieved between about 10 hours and about 48 hours after administration. After the maximal exposure level is achieved the compositions may achieve a sustained rate of release whereby a substantial percentage of the maximal level is obtained for a period of time. For example, the sustained rate may about 50%, about 60%, about 70%, about 80%, about 90% or about 100% of the maximal exposure level. Exemplary periods of time for maintaining the sustained rate are about 3 days, about 4 days, about 5 days, about 6 days, about 1 week, about 2 weeks, about 4 weeks, about 6 weeks, or about 8 weeks, after the maximal exposure level is achieved. Subsequently, the sustained rate may lower to a reduced exposure rate. Such reduced exposure rates may be about 5%, about 10%, about 20%, about 30%, about 40%, about 50% or about 60% of the maximal exposure level. For example, in one embodiment (PE0256) a maximal exposure level of 1000 ng/mL is obtained within about 1-2 days. After this period, a sustained rate of about 70-100% of the maximal exposure level is maintained until about days 10-12 whereupon a reduced exposure rate from about 60% decreasing down to about 10% is obtained for the remainder of the study.

In various embodiments, the plasma concentration of the active agent does not change by more than a factor of about 20, or a factor of about 10, or a factor of about 5, or a factor of about 3 over the course of a plurality of administrations, such as at least 2, at least about 5, or at least about 10 administrations of the formulation. In some embodiments, the plasma concentration of the active agent does not change by more than a factor of about 20, or a factor of about 10, or a factor of about 5, or a factor of about 3 between each administration. In some embodiments, there is some accumulation until steady state is reached (e.g. after about 3 to about 4 administrations). The administrations are substantially evenly spaced, such as, for example, about daily, or about once per week, or from one to about five times per month, or about once every two months, or about once every three months. In other embodiments, the dose may be steadily increased over several administrations, so steady state is reached after 5 or more administrations.

The pharmaceutical compositions disclosed herein may be administered in smaller doses and/or less frequently than unfused or unconjugated counterparts. While one of skill in the art can determine the desirable dose in each case, a suitable dose of the therapeutic agent for achievement of therapeutic benefit, may, for example, be in a range of about 1 microgram (μg) to about 100 milligrams (mg) per kilogram body weight of the recipient per dose, preferably in a range of about 10 μg to about 50 mg per kilogram body weight per dose and most preferably in a range of about 10 μg to about 50 mg per kilogram body weight per dose. In some embodiments, the pharmaceutical composition is administered at a low dose. In some embodiments, the pharmaceutical composition is administered at a dose between 1 mg per kilogram per body weight per dose to about 9 mg per kilogram per body weight per dose. In some embodiments, the pharmaceutical composition is administered at about 1 mg per kilogram body weight per dose, about 3 mg per kilogram body weight per dose, and/or about 9 mg per kilogram body weight per dose. The desired dose may be presented as one dose or two or more sub-doses administered at appropriate intervals throughout the day. These sub-doses can be administered in unit dosage forms, for example, containing from about 10 μg to about 1000 mg, preferably from about 50 μg to about 500 mg, and most preferably from about 50 μg to about 250 mg of active ingredient per unit dosage form. Alternatively, if the condition of the recipient so requires, the doses may be administered as a continuous infusion.

In certain embodiments, the subject is a human, but in other embodiments may be a non-human mammal, such as a domesticated pet (e.g., dog or cat), or livestock or farm animal (e.g., horse, cow, sheep, or pig).

### Co-Administration

The compositions of the present disclosure may be co-administered with any appropriate therapeutic, vitamin, or diet. In some embodiments, the compositions of the present disclosure are administered concomitantly with another therapeutic, vitamin, and/or diet. In some embodiments, the compositions of the present disclosure are administered before or after another therapeutic, vitamin, and/or diet. In some embodiments, the compositions of the present disclosure are administered on the same day as another therapeutic, vitamin, and/or diet. In some embodiments, the compositions of the present disclosure are administered on a different day from another therapeutic, vitamin, and/or diet.

Examples of therapeutics, vitamins, and/or diets that may be administered with the compositions of the invention, include, but are not limited to, oral calcium, vitamin D, active vitamin D metabolites, vitamin D analogs (e.g. 1α-hydroxyvitamin D (alfacalcidol), dihydrotachysterol), vitamin D2, vitamin D3, calcium carbonate, calcium citrate, zoledronic acid, bisphosphonate, selective estrogen receptor modulators (e.g. raloxifine), anti-resorptive therapy, alendronate, estrogen, anabolic agents, cathepsin K inhibitors (e.g. odanacatib), anti-sclerostin antibodies (e.g. romosozumab), thiazide diuretic therapy, hydrochlorothiazide, chlorthalidone, potassium supplementation, potassium and magnesium-sparing diuretics, and phosphate binders,

In some embodiments, a composition of the disclosure is administered before a bisphosphonate. In some embodiments, co-administration of an instant composition and a bisphosphonate has a synergistic effect on increase bone mineral density.

### Patient Populations

In some embodiments, any patient diagnosed with, susceptible to, or at risk of a disease associated with loss of bone density and/or increased bone fracture, can be treated with the compositions of the disclosure. In some embodiments, the disease associated with loss of bone density and/or increased risk of bone fracture is osteoporosis or hypoparathyroidism. In some embodiments, the hypoparathyroidism is selected from the group including, but not limited to, congenital hypoparathyroidism, acquired hypoparathyroidism, autoimmune hypoparathyroidism, and idiopathic hypoparathyroidism.

In some embodiments, patients who can be treated with the compositions of the instant disclosure include, but are not limited to, post-menopausal women and perimenopausal women.

It should be understood that singular forms such as “a,” “an,” and “the” are used throughout this application for convenience, however, except where context or an explicit statement indicates otherwise, the singular forms are intended to include the plural. All numerical ranges should be understood to include each and every numerical point within the numerical range, and should be interpreted as reciting each and every numerical point individually. The endpoints of all ranges directed to the same component or property are inclusive, and intended to be independently combinable.

The term “about” when used in connection with a referenced numeric indication means the referenced numeric indication plus or minus up to 10% of that referenced numeric indication. For example, the language “about 50” covers the range of 45 to 55.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

As used herein, “half-life” (which generally refers to in vivo half-life or circulatory half-life) is the period of time that is required for a 50% diminution of bioactivity of the active agent to occur. In some embodiments, this term includes both prolonged exposure and a long half-life (e.g. both a slow uptake from the injection site and retardation of clearance compared to the unconjugated peptide).

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein.

### EXAMPLES

### Example 1

The in vitro potency of fusion proteins comprising PTH, PTHrP or analogues thereof can be determined by a variety of assays known in the art. One assay system detects cAMP generated upon activation of the receptor, for example the PTH1R.

Potency was demonstrated utilizing a Eurofins cAMP Hunter™ eXpress PTHR1 CHO-K1 GPCR assay kit (Eurofins DiscoverX Corporation, Fremont, Calif. Catalogue number 95-0118E2CP2S). Chinese Hamster Ovary (CHO-K1) cells engineered to overexpress parathyroid hormone receptor 1 (PTH1R) were used to measure receptor activation based on the resultant cAMP production within the cells. When the receptor becomes activated, it causes generation of cAMP within the cells. The cells are lysed and the amount of cellular cAMP is measured via a gain-of-signal competitive immunoassay based on proprietary enzyme fragment complementation (EFC) technology. EFC technology uses β-galactose enzyme split into two fragments (enzyme donor and enzyme acceptor) that, independently, have no activity but in solution, combine to form active β-galactose enzyme. cAMP from lysed cells and enzyme donor-labeled cAMP (ED-cAMP) compete to bind to anti-cAMP antibody. The unbound ED-cAMP is free to complement the enzyme acceptor fragment and form the active β-galactose enzyme, which produces a luminescence signal directly proportional to the amount of cAMP in the cells.

The day prior to the assay, eXpress CHO-K1 PTHR1 cells were plated on a 96-well tissue culture plate and incubated overnight at 37° C., 5% CO₂. The following day, serial dilutions of PTH constructs were prepared in cell assay buffer. The cell plating reagent was removed from the cells and replaced with cell assay buffer. Serial dilutions of the PTH constructs or control PTH 91-34) peptide were then added to the plate in duplicate and the plate was incubated at 37° C., 5% CO₂ for 30 minutes. cAMP antibody was then added to each well followed by a solution of lysis buffer, substrate reagent and enzyme donor labeled cAMP (ED-cAMP). The plate was incubated 1 hr at room temperature. A solution of enzyme acceptor (EA) fragment was added to each well and the plate was incubated 3 hr at room temperature. The free ED-cAMP (not bound to anti-cAMP antibody) combines with the EA fragment to form the active enzyme, which hydrolyzes the substrate to produce the luminescent signal. The plate was then read on a luminescence plate reader at 1 sec/well.

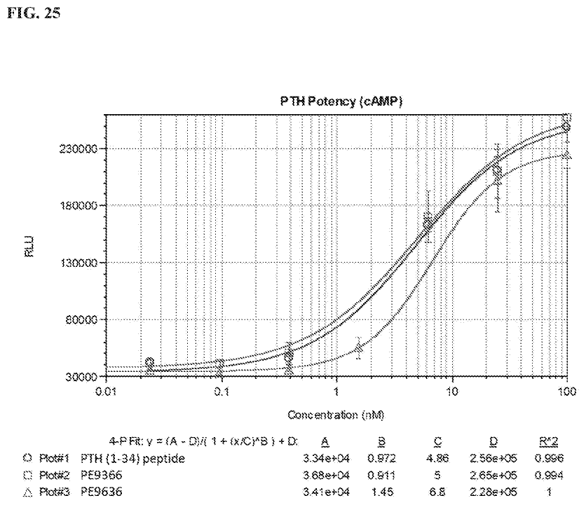

Constructs PE9366 and PE9636 showed similar potency to PTH (1-34) peptide (FIG. 25). In a separate analysis, PE9356 showed similar potency to PE9366 and PE9636 using the cAMP assay.

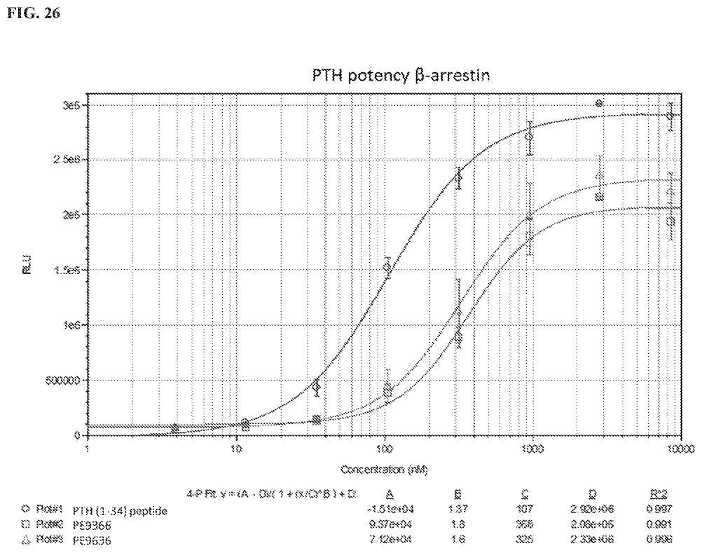

An alternative assay used a Eurofins (DiscoverX) PathHunter eXpress PTHR1 CHO-K1 β-Arrestin GPCR Assay kit (Eurofins DiscoverX Corporation, Fremont, Calif. Catalogue number 93-0315E2CPOS), which determines relatively potency based on the recruitment of β-arrestin 2 following binding to the receptor. In this assay, PE9366 and PE9636 showed similar, but slightly reduced potency compared to PTH (1-34) peptide (FIG. 26).

The ability of PE9366 and PE9636 to activate the PTHR2 receptor was evaluated using a Eurofins (DiscoverX) PathHunter eXpress PTHR2 CHO-K1 β-Arrestin GPCR Assay kit (Catalogue number 93-0306E2CP0S). The data showed that both PE9366 and PE9636 were approximately 10-20 fold less potent than PTH (1-34), indicating selectivity for PTHR1 over PTHR2.

PE9366 and PE9636 were formulated at either pH 4.0 (6.8 mM Glacial Acetic Acid, 1.2 mM Sodium Acetate Anhydrous, 28 mM Metacresol, 249 mM D-Mannitol ACS) or pH 6.0 (14 mM L-Histidine, 11 mM L-Histidine HCL, 110 mM NaCl). In both instances, there was no reduction in potency after storage at room temperature for 3 weeks as determined using a Eurofins cAMP Hunter™ eXpress PTHR1 CHO-K1 GPCR assay kit (Eurofins DiscoverX Corporation, Fremont, Calif. Catalogue number 95-0118E2CP2S), nor any evidence of product breakdown when analyzed by SDS PAGE.

### INCORPORATION BY REFERENCE

All patents and publications referenced herein are hereby incorporated by reference in their entireties, including the publications disclosed below. The publications discussed herein are provided solely for their disclosure prior to the filing date of the present application. Nothing herein is to be construed as an admission that the present disclosure is not entitled to antedate such publication by virtue of prior disclosure.

### REFERENCES

Sato et al. (2016) Journal of Investigative Dermatology. 136, 1449e1459.
Dean et al. (2008) Molecular Endocrinology. 22: 156-166.
Augustin and Horwitz. (2013) Curr Osteoporos Rep. December; 11(4): doi:10.1007/s11914-013-0171-2.
Bilezikian et al. J Clin Endocrinol Metab, June 2016, 101(6):2313-2324
Black et al. J Clin Endocrinol Metab. June 2008, 93(6):2166-2172.
Chan et al. British Medical Bulletin, 2016, 119:129-141
Clarke et al. Clinical Therapeutics/Volume 36, Number 5, 2014: 722-736
Fujita et al. Osteoporos Int (1999) 9:296-306
Gardella and Vilardaga. Pharmacol Rev. 2015; 67(2):310-37.
Gonnelli and Caffarelli. Clinical Cases in Mineral and Bone Metabolism 2016; 13(2):106-109
Greenspan et al. Ann Intern Med. 2007; 146:326-339
Hass and LeBoff (2018) Journal of the Endocrine Society. Vol. 2, Iss. 8:922-932
Hattersley et al. Endocrinology, January 2016, 157(1):141-149
Kim and Keating. Drugs (2015) 75:1293-1303
Leder Curr Osteoporos Rep (2017) 15:110-119
Mannstadt et al. Lancet Diabetes Endocrinol. 2013 December; 1(4):275-83.
Miller et al. JAMA. 2016; 316(7):722-733.
Rubin et al. Journal of Bone and Mineral Research, Vol. 31, No. 5, May 2016, pp 1082-1088.
Shimizu et al. J Bone Miner Res. 2016 July; 31(7): 1405-1412.
Silva and Bilezikian. Curr Opin Pharmacol. 2015 June; 22: 41-50.
Horizonte et al. Curr Opin Pharmacol. 2015 June; 22: 41-50.
Tella S, Kommalapati A, Correa R (May 31, 2017) Cureus 9(5): e1300. DOI 10.7759/cureus.1300
Winer et al. J Clin Endocrinol Metab. 2012 February; 97(2): 391-399.
Winer et al. J Pediatr. 2014 September; 165(3): 556-563.

What is claimed is:

1. A pharmaceutical composition comprising a parathyroid hormone (PTH) or a parathyroid hormone related protein (PTHrP) and an elastin-like peptide, wherein the elastin-like peptide (ELP) comprises repeating units of any one of SEQ ID NOs: 1-13.

2. The pharmaceutical composition of claim 1, wherein the PTH is a full-length PTH (SEQ ID NO: 14).

3. The pharmaceutical composition of claim 1, wherein the PTH is a truncated PTH.

4. The pharmaceutical composition of claim 3, wherein the PTH is truncated at the C-terminus.

5. The pharmaceutical composition of claim 4, wherein the PTH is PTH(1-34) (SEQ ID NO: 16) or PTH(1-32) (SEQ ID NO: 18).

6. The pharmaceutical composition of claim 1, wherein the PTHrP is a full-length PTHrP.

7. The pharmaceutical composition of claim 1, wherein the PTHrP is a truncated PTHrP.

8. The pharmaceutical composition of claim 7, wherein the PTHrP is truncated at the C-terminus.

9. The pharmaceutical composition of claim 8, wherein the PTHrP is PTHrP (1-32).

10. The pharmaceutical composition of claim 1, wherein the ELP comprises structural repeats of SEQ ID NO: 3.

11. The pharmaceutical composition of claim 10, wherein X is independently selected from valine, glycine, and alanine.

12. The pharmaceutical composition of claim 11, wherein X is independently selected from V, G and A at a ratio of 5:3:2.

13. The pharmaceutical composition of claim 10, wherein X is independently selected from valine and alanine.

14. The pharmaceutical composition of claim 13, wherein X is independently selected from valine and alanine at a ratio of 6:3.

15. The pharmaceutical composition of any of claims 1 to 14, wherein the ELP comprises at least 90 repeating structural units.

16. The pharmaceutical composition of claim 15, wherein the ELP comprises at least 120 repeating structural units.

17. The pharmaceutical composition of claim 16, wherein the ELP comprises the amino acid sequence of SEQ ID NO: 34.

18. The pharmaceutical composition of claim 16, wherein the ELP comprises at least 144 repeating structural units.

19. The pharmaceutical composition of claim 18, wherein the ELP comprises the amino acid sequence of SEQ ID NO: 35.

20. The pharmaceutical composition of any of claims 1 to 18, wherein the PTH or PTHrP and ELP are in a fusion protein.

21. The pharmaceutical composition of claim 20, wherein the fusion protein is selected from:
a) SEQ ID NO: 20;
b) SEQ ID NO: 22;
c) SEQ ID NO: 24;
d) SEQ ID NO: 26;
e) SEQ ID NO: 28; and
f) SEQ ID NO: 30.

22. A method of increasing serum calcium levels in a subject comprising administering to a subject in need thereof the pharmaceutical composition of any one of claims 1-21.

23. A method of increasing bone formation in a subject, comprising to a subject in need thereof administering the pharmaceutical composition of any one of claims 1 to 21.

24. A method of reducing bone absorption in a subject, comprising administering to a subject in need thereof the pharmaceutical composition of any one of claims 1 to 21.

25. A method of treating, ameliorating, or delaying osteoporosis, comprising administering to a subject in need thereof the pharmaceutical composition of any one of claims 1 to 21.

26. A method of treating, ameliorating, or delaying hypoparathyroidism, comprising administering to a subject in need thereof the pharmaceutical composition of any one of claims 1 to 21.

27. The method of any of claims 22 to 26, wherein the pharmaceutical composition is administered subcutaneously.

28. The method of any of claims 22 to 27, wherein the pharmaceutical composition is administered daily or weekly.
